SEQUENCE LISTING (27 sequences)

SEQ ID NO: 1 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 2 - protein, 556 aa, Arabidopsis thaliana
MTTQDVIVNDQNDQKQCSNDVIFRSRLPDIYIPNHLPLHDYIFENISEFAAKPCLINGPTGEVYTYADVHVTSRKLAAGLHNLGVKQHDVVMILLPNSPEVVLTFLAASFIGAITTSANPFFTPAEISKQAKASAAKLIVTQSRYVDKIKNLQNDGVLIVTTDSDAIPENCLRFSELTQSEEPRVDSIPEKISPEDVVALPFSSGTTGLPKGVMLTHKGLVTSVAQQVDGENPNLYFNRDDVILCVLPMFHIYALNSIMLCSLRVGATILIMPKFEITLLLEQIQRCKVTVAMVVPPIVLAIAKSPETEKYDLSSVRMVKSGAAPLGKELEDAISAKFPNAKLGQGYGMTEAGPVLAMSLGFAKEPFPVKSGACGTVVRNAEMKILDPDTGDSLPRNKPGEICIRGNQIMKGYLNDPLATASTIDKDGWLHTGDVGFIDDDDELFIVDRLKELIKYKGFQVAPAELESLLIGHPEINDVAVVAMKEEDAGEVPVAFVVRSKDSNISEDEIKQFVSKQVVFYKRINKVFFTDSIPKAPSGKILRKDLRARLANGLMN

SEQ ID NO: 3 - protein, 310 aa, Saccharomyces cerevisiae
MPITIKSRSKGLRDTEIDLSKKPTLDDVLKKISANNHNISKYRIRLTYKKESKQVPVISESFFQEEADDSMEFFIKDLGPQISWRLVFFCEYLGPVLVHSLFYYLSTIPTVVDRWHSASSDYNPFLNRVAYFLILGHYGKRLFETLFVHQFSLATMPIFNLFKNCFHYWVLSGLISFGYFGYGFPFGNAKLFKYYSYLKLDDLSTLIGLFVLSELWNFYCHIKLRLWGDYQKKHGNAKIRVPLNQGIFNLFVAPNYTFEVWSWIWFTFVFKFNLFAVLFLTVSTAQMYAWAQKKNKKYHTRRAFLIPFVF

SEQ ID NO: 4 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 5 - protein, 392 aa, Vitis vinifera
MASVEEFRNAQRAKGPATILAIGTATPDHCVYQSDYADYYFRVTKSEHMTELKKKFNRICDKSMIKKRYIHLTEEMLEEHPNIGAYMAPSLNIRQEIITAEVPRLGRDAALKALKEWGQPKSKITHLVFCTTSGVEMPGADYKLANLLGLETSVRRVMLYHQGCYAGGTVLRTAKDLAENNAGARVLVVCSEITVVTFRGPSEDALDSLVGQALFGDGSSAVIVGSDPDVSIERPLFQLVSAAQTFIPNSAGAIAGNLREVGLTFHLWPNVPTLISENIEKCLTQAFDPLGISDWNSLFWIAHPGGPAILDAVEAKLNLEKKKLEATRHVLSEYGNMSSACVLFILDEMRKKSLKGEKATTGEGLDWGVLFGFGPGLTIETVVLHSVPTVTN

SEQ ID NO: 6 - protein, 392 aa, Vitis pseudoreticulata
MASVEEIRNAQRAKGPATILAIGTATPDHCVYQSDYADYYFRVTKSEHMTELKKKFNRICDKSMIKKRYIHLTEEMLEEHPNIGAYMAPSLNIRQEIITAEVPKLGKEAALKALKEWGQPKSKITHLVFCTTSGVEMPGADYKLANLLGLETSVRRVMLYHQGCYAGGTVLRTAKDLAENNAGARVLVVCSEITVVTFRGPSEDALDSLVGQALFGDGSAAVIVGSDPDISIERPLFQLVSAAQTFIPNSAGAIAGNLREVGLTFHLWPNVPTLISENIENCLTKAFDPIGISDWNSLFWIAHPGGPAILDAVEAKVGLDKQKLKATRHILSEYGNMSSACVLFILDEMRKKSLKEGKTTTGEGLDWGVLFGFGPGLTIETVVLHSVGTDSN

SEQ ID NO: 7 - protein, 389 aa, Arachis hypogaea
MVSVSGIRKVQRAEGPATVLAIGTANPPNCVDQSTYADYYFRVTNSEHMTDLKKKFQRICERTQIKNRHMYLTEEILKENPNMCAYKAPSLDAREDMMIREVPRVGKEAATKAIKEWGQPMSKITHLIFCTTSGVALPGVDYELIVLLGLDPSVKRYMMYHQGCFAGGTVLRLAKDLAENNKDARVLIVCSENTSVTFRGPSETDMDSLVGQALFADGAAAIIIGSDPVPEVENPLFEIVSTDQQLVPNSHGAIGGLLREVGLTFYLNKSVPDIISQNINDALSKAFDPLGISDYNSIFWIAHPGGRAILDQVEEKVNLKPEKMKATRDVLSNYGNMSSACVFFIMDLMRKKSLEAGLKTTGEGLDWGVLFGFGPGLTIETVVLRSMAI

SEQ ID NO: 8 - protein, 393 aa, Pinus densiflora
MGGVDFEGFRKLQRADGFASILAIGTANPPNAVDQSTYPDYYFRITGNEHNTELKDKFKRICERSAIKQRYMYLTEEILKKNPDVCAFVEVPSLDARQAMLAMEVPRLAKEAAEKAIHEWGQSKSGITHLIFCSTTTPDLPGADFEVAKLLGLHPSVKRVGVFQHGCFAGGTVLRLAKDLAENNRGARVLVICSETTAVTFRGPSETHLDSLVGQALFGDGASALIVGADPIPQVEKACFEIVRTSQTVVPNSDGAIGGKVREVGLTFQLKGAVPDLISANIENCLVEAFSQFKICDWNKLFWVVHPGGRAILDRVEAKLNLDPTKLIPTRHVMSEYGNMSSACVHFILDETRKASLRNGCSTTGEGLEMGVLFGFGPGLTIETVVLKSVPLQ

SEQ ID NO: 9 - protein, 396 aa, Pinus strobus
MSVGMGVDLEAFRKSQRADGFASILAIGTANPPNVVDQSTYPDYYFRNTNNEDNTDLKDKFKRICERSAIKKRHMYLTEEILKKNPELCAFLEVPSLDTRQAMLAVEVPRLGKEAAEKAIEEWGQPKSRITHLIFCTTTTPDLPGADFEVAKLLGLHPSVKRVGVFQHGCFAGGTVLRLAKDLAENNRGARVLVVCSENTAVTFRGPSETHLDGLVGLALFGDGAAALIVGADPIPQVEKPCFEIVWTAQTVVPNSDGAISGKLREVGLTFQLKGAVPDLISTNIEKCLVEAFSQFNISDWNQLFWIAHPGGRAILDQVEASLNLDPTKLRATRHVMSEYGNMSSACVHFILDETRKASRQNGCSTSGGGFQMGVLFGFGPGLTVETVVLKSIPFP

SEQ ID NO: 10 - protein, 392 aa, Pinus sylvestris
MGGVDFEGFRKLQRADGFASILAIGTANPPNAVDQSTYPDFYFRITGNEHNTELKDKFKRICERSAIKQRYMYLTEEILKKNPDVCAFVEVPSLDARQAMLAMEVPRLAKEADEKAIQEWGQSKSGITHLIFCSTTTPDLPGADFEVAKLLGLHPSVKRVGVFQHGCFAGGTVLRMAKDLAENNRGARVLVICSETTAVTFRGPSETHLDSLVGQALFGDGACALIVGADPIPQVEKACFEIVWTAQTVVPNSEGAIGGKVREVGLTFQLKGAVPDLISANIENCMVEAFSQFKISDWNKLFWVVNPGGPAILDRVEAKLNLDPTKLIPTRHVMSEYGNMSSACFHFILDQTRKASLENGFSTTGEGLEMGVLFGFGGLTIETVVLKSVPIQ

SEQ ID NO: 11 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 12 - protein, 525 aa, Saccharomyces cerevisiae
MDQLVKTEVTKKSFTAPVQKASTPVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 13 - protein, 326 aa, Cannabis sativa
MKNSIRAATTNQTEPPESDNHSVATKILNFGKACWKLQRPYTIIAFTSCACGLFGKELLHNTNLISWSLMFKAFFFLVAVLCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 14 - protein, 323 aa, Cannabis sativa
MKSSIKAHGEIEADGSNGTSEFNVMKSGNAIWRFVRPYAAKGVLFNSAAMFAKELVGNLNLFSWPLMFKILSFTLVILCIFVSTSGINQIYDLDIDRLNKPNLPVASGEISVELAWLLTIVCTISGLTLTIITNSGPFFPFLYSASIFFGFLYSAPPFRWKKNPFTACFCNVMLYVGTSVGVYYACKASLGLPANWSPAFCLLFWFISLLSIPISIAKDLSDIEGDRKFGIITFSTKFGAKPIAYICHGLMLLNYVSVMAAAIIWPQFFNSSVILLSHAFMAIWVLYQAWILEKSNYATETCQKYYIFLWIIFSLEHAFYLFM

SEQ ID NO: 15 - protein, 327 aa, Cannabis sativa
MSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 16 - protein, 322 aa, Cannabis sativa
MRNSIRACAEAGAAGSNPVLNKVSDFRDACWRFLRPHTIRGTTLGSIALVARALIENPNLIKWSLLLKAFSGLLALICGNGYIVGINQIYDIGIDKVNKPYLPIAAGDLSVQSAWYLVILFAVAGLLTVGFNFGPFITSLYCLGLVLGTIYSVPPFRMKRFPVAAFLIIATVRGFLLNFGVYYATRAALGLTFEWSSAVAFITTFVTLFALVIAITKDLPDVEGDRKFQISTFATKLGVRNIAYLGSGLLLLNYIGAIAAAIYMPQAFKRNLMLPIHTILALSLVFQAWVLEQANYTKEAIAGFYRFIWNLFYVEYIIFPFI

SEQ ID NO: 17 - protein, 327 aa, Cannabis sativa
MKNSIRTDTANQTEPPESNTKYSVVTKILSFGHTCWKLQRPYTFIGVISCACGLFGRELFHNTNLLSWSLMLKAFSSLMVILSVNLCTNIINQITDLDIDRINKPDLPLASGEMSIETAWIMSIIVALTGLILTIKLNCGPLFISLYCVSILVGALYSVPPFRWKQNPNTAFSSYFMGLVIVNFTCYYASRAAFGLPFEMSPPFTFILAFVKSMGSALFLCKDVSDIEGDSKHGISTLATRYGAKNITFLCSGIVLLTYVSAILAAIIWPQAFKSNVMLLSHATLAFWLIFQTREFALTNYNPEAGRKFYEFMWKLHYAEYLVYVFI

SEQ ID NO: 18 - protein, 326 aa, Humulus lupulus
MGNSIRASAQIEDRPPESGNLSALTNVKDFVSVCWEYVRPYTAKGVIICSSCLFGRELLENPNLFSWPLIFRALLGMLAILGSCFYTAGINQIFDMDIDRINKPDLPLVSGRISVESAWLLTLSPAIIGFILILKLNSGPLLTSLYCLAILSGTIYSVPPFRWKKNPITAFLCILMIHAGLNFSVYYASRAALGLAFVWSPSFSFITAFITFMTLTLASSKDLSDINGDRKFGVETFATKLGAKNITLLGTGLLLLNYVAAISTAIIWPKAFKSNIMLLSHAILAFSLFFQARELDRTNYTPEACKSFYEFIWILFSAEYVVYLFI

SEQ ID NO: 19 - protein, 326 aa, Humulus lupulus
MGNSIRASAQIEDRPPESGNLSALTNVKDFVSVCWEYVRPYTAKGVIICSSCLFGRELLENPNLFSRPLIFRALLGMLAILGSCFYTAGINQIFDMDIDRINKPDLPLVSGRISVESAWLLTLSPAIIGFILILKLNSGPLLTSLYCLAILSGTIYSVPPFRWKKNPITAFLCILMIHAGLNFSVYYASRAALGLAFAWSPSFSFITAFITFMTLTLASSKDLSDINGDRKFGVETFATKLGAKNITLLGTGLLLLNYVAAISTAIIWPKAFKSNIMLLSHAILAFSLIFQARELDRTNYTPEACKSFYEFIWILFSAEYVVYLFI

SEQ ID NO: 20 - protein, 324 aa, Humulus lupulus
MPNSLTAWSHQSEFPSTIVTKGSNFGHASWKFVRPIPFVAVSIICTSLFGAELLKNPNLFSWQLMFDAFQGLVVILLYHIYINGLNQIYDLESDRINKPDLPLAAEEMSVKSAWFLTIFSAVASLLLMIKLKCGLFLTCMYCCYLVIGAMYSVPPFRWKMNTFTSTLWNFSEIGIGINFLINYASRATLGLPFQWRPPFTFIIGFVSTLSIILSILKDVPDVEGDKKVGMSTLPVIFGARTIVLVGSGFFLLNYVAAIGVAIMWPQAFKGYIMIPAHAIFASALIFKTWLLDKANYAKEASDSYYHFLWFLMIAEYILYPFIST

SEQ ID NO: 21 - protein, 541 aa, Cannabis sativa
MIFDGTTMSIAIGLLSTLGIGAEANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 22 - protein, 540 aa, Cannabis sativa
MIFDGTTMSIAIGLLSTLGIGAEANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 23 - protein, 214 aa, Cannabis sativa
MATSDGSSNAATKEEAVQVEPKTGISFPVKLDDGKILYCVGYNKKSLLGLSIKAYGFGLYVDSDKLKDVLKSKIEKAPSKPTEEMYQLAIDGDFGMTIKMVVSFSGVKLSMAKKGFTEAMRESMKKLTGQKNEELSNKVFGTTSDKIKLRLGSEMIVSKLPGYVLETKVNGELVSRVESELLCRAYFRNYLGEDTLECEKESREMFGQSMLSLF

SEQ ID NO: 24 - protein, 209 aa, Humulus lupulus
MENNMVMVHEIPFPPEIKTTKPLSLLGYGITDMEIHFLQVKFTAIGVYLDSDVVKHLQQWKGKKGNELAEDDDFFDALISAPVEKYLRIVVIKEIKGSQYGVQLESSVRDRLAAEDMYEEEEEAALEKIVEFLQSKYFKKDTLITFHFPATSPTAEIVVTLEGKEESKLKVENKNVVDMIKKWYLGGTSGVSPSTISSLADNLSAELSK

SEQ ID NO: 25 - protein, 703 aa, Zea mays
MAGNGAIVESDPLNWGAAAAELAGSHLDEVKRMVAQARQPVVKIEGSTLRVGQVAAVASAKDASGVAVELDEEARPRVKASSEWILDCIAHGGDIYGVTTGFGGTSHRRTKDGPALQVELLRHLNAGIFGTGSDGHTLPSEVTRAAMLVRINTLLQGYSGIRFEILEAITKLLNTGVSPCLPLRGTITASGDLVPLSYIAGLITGRPNAQAVTVDGRKVDAAEAFKIAGIEGGFFKLNPKEGLAIVNGTSVGSALAATVMYDANVLAVLSEVLSAVFCEVMNGKPEYTDHLTHKLKHHPGSIEAAAIMEHILDGSSFMKQAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSADEHNQDVNSLGLVSARKTAEAIDILKLMSSTYIVALCQAVDLRHLEENIKASVKNTVTQVAKKVLTMNPSGELSSARFSEKELISAIDREAVFTYAEDAASASLPLMQKLRAVLVDHALSSGERGAGALRVLQDHQVRGGAPRGAAPGGGGRPRGVAEGTAPVANRIADSRSFPLYRFVREELGCVFLTGERLKSPGEECNKVFVGISQGKLVDPMLECLKEWDGKPLPINIK

SEQ ID NO: 26 - protein, 506 aa, Ammi majus
MMDFVLLEKALLGLFIATIVAITISKLRGKKLKLPPGPIPVPVFGNWLQVGDDLNQRNLVEYAKKFGDLFLLRMGQRNLVVVSSPDLAKDVLHTQGVEFGSRTRNVVFDIFTGKGQDMVFTVYSEHWRKMRRIMTVPFFTNKVVQQYRFGWEDEAARVVEDVKANPEAATNGIVLRNRLQLLMYNNMYRIMFDRRFESVDDPLFLKLKALNGERSRLAQSFEYNFGDFIPILRPFLRGYLKLCQEIKDKRLKLFKDYFVDERKKLESIKSVGNNSLKCAIDHIIEAQEKGEINEDNVLYIVENINVAAIETTLWSIEWGIAELVNNPEIQKKLRHELDTVLGAGVQICEPDVQKLPYLQAVIKETLRYRMAIPLLVPHMNLHEAKLAGYDIPAESKILVNAWWLANNPAHWNKPDEFRPERFLEEESKVEANGNDFKYIPFGVGRRSCPGIILALPILGIVIGRLVQNFELLPPPGQSKIDTAEKGGQFSLQILKHSTIVCKPRSS

SEQ ID NO: 27 - protein, 691 aa, Saccharomyces cerevisiae
MPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELHLDEQEAKFTSQFQYTVLNEITDSMSLGEPSAHYLPSHQLNRNADGIQLGPFDLSQPYIAPIVKSRELFSSNDRNCIHSEFDLSGSNIKYSTGDHLAVWPSNPLEKVEQFLSIFNLDPETIFDLKPLDPTVKVPFPTPTTIGAAIKHYLEITGPVSRQLFSSLIQFAPNADVKEKLTLLSKDKDQFAVEITSKYFNIADALKYLSDGAKWDTVPMQFLVESVPQMTPRYYSISSSSLSEKQTVHVTSIVENFPNPELPDAPPVVGVTTNLLRNIQLAQNNVNIAETNLPVHYDLNGPRKLFANYKLPVHVRRSNFRLPSNPSTPVIMIGPGTGVAPFRGFIRERVAFLESQKKGGNNVSLGKHILFYGSRNTDDFLYQDEWPEYAKKLDGSFEMVVAHSRLPNTKKVYVQDKLKDYEDQVFEMINNGAFIYVCGDAKGMAKGVSTALVGILSRGKSITTDEATELIKMLKTSGRYQEDVW

(19) United States
(12) Patent Application Publication
(10) Pub. No.: US 2023/0257785 A1
(43) Pub. Date: Aug. 17, 2023

(54) PRODUCTION OF BIOACTIVE BIBENZYLIC ACID OR DERIVATIVES THEREOF BY GENETICALLY MODIFIED MICROBIAL HOSTS

(71) Applicants: BARRIT SARL, Huningue (FR); IPTECTOR ASSETS APS, Kgs. Lyngby (DK)

(72) Inventor: Michael NAESBY, Huningue (FR)

(73) Assignees: BARRIT SARL, Huningue (FR); IPTECTOR ASSETS APS, Kgs. Lyngby (DK)

(21) Appl. No.: 17/758,574

(22) PCT Filed: Jan. 9, 2021
(86) PCT No.: PCT/EP2021/050318

§ 371 Date: Jul. 10, 2022

(30) Foreign Application Priority Data
Jan. 10, 2020 (EP) 20151327.2

(51) Int. Cl. C12P 7/42; C12N 15/81; C12N 9/90; C12N 1/16; C12N 9/02; C12N 9/10; C12N 9/00; C12N 9/88

(52) U.S. Cl. CPC C12P 7/42; C12N 15/81; C12N 9/90; C12N 1/16; C12N 9/0071; C12N 9/1029; C12N 9/93; C12N 9/001; C12N 9/1085; C12N 9/0042; C12N 9/88; C12Y 403/01023; C12Y 505/01006; C12Y 205/01093; C12Y 106/02004; C12Y 602/01012; C12Y 403/01024; C12Y 103/01009; C12Y 404/01026

(57) ABSTRACT

The present invention relates to a genetically modified host cell producing a bibenzylic acid or a derivative thereof expressing a) one or more genes encoding a polyketide synthase (PKS); b) one or more genes encoding a polyketide cyclase (PKC); and c) one or more genes encoding a double bond reductase (DBR); and one or more genes encoding polypeptides selected from d) a tyrosine ammonia lyase polypeptide (TAL); e) a phenylalanine ammonia lyase polypeptide (PAL); f) a cinnamate 4-hydroxylase polypeptide (C4H); g) a cytochrome p450 reductase polypeptide (CPR); h) a 4-coumarate-CoA ligase polypeptide (4CL); and/or i) a non-catalytic chalcone isomerase type III or IV polypeptide (CHIL); wherein the at least one gene is heterologous to the host cell.

### FIELD OF THE INVENTION

The present invention relates to genetically modified microorganisms producing bibenzylic acids or derivatives thereof using an operative metabolic pathway, comprising a double bond reductase (DBR), capable of reducing the C2-C3 alkene double bond of phenylpropanoid precursors, a polyketide synthase (PKS) capable of producing a tetraketide derived from the reduced phenylpropanoid, a polyketide cyclase (PKC), capable of producing a bibenzylic acid (BBA) from said tetraketide, a prenyltransferase (PT) capable of transferring a geranyl to the BBA, producing a bibenzylgerolic acid (BBGA), and a cyclase capable of cyclizing said geranylated BBGA to yield perrottetitenoic acid (PETA), which spontaneously or after induction by heat, yields perrottetinene (PET). It includes methods of producing BBAs using such microorganisms, comprising the biosynthetic pathway enzymes, and to compositions, comprising BBA and its derivatives, resulting from such methods. It further includes the use of BBA and derivatives as modulators of the endocannabinoid system, including receptors 1 and 2 (CB1 and CB2).

### BACKGROUND OF THE INVENTION

Bibenzylic acids (BBA) form a distinct group of plant metabolites. The core structure is related to the better-known stilbenes and dihydrostilbenes, but as the name BBA suggests, they comprise a carboxylic acid group which, in the case of stilbenes, is normally lost during biosynthesis and ring closure of the second ring. BBA derivatives are found in various plants, e.g. Pidgeon pea (Cajanus cajan) and Hortensia (Hydrangea macrophylla), and the corresponding dihydro-versions have been found in several liverworts, including the genus Radula (Chicca 2019), as well as in the two legumes Glycyrrhiza foetida and Amorpha fruticosa (Weidner 2012) the latter of which has given name to the BBA group of amorfrutins. A geranylated BBA was recently discovered in Radula perrottetii and was named perrottetinene (PET). Interestingly, the chemical structure of PET and its precursors share a high similarity to structures of the cannabinoid biosynthetic pathway (FIG. 3). Hence, the two groups, cannabinoids (CAN) and perrottetinoids (PET), may also share several similarities in their biosynthetic pathways. One group (CAN) has the basic structural features of olivetolic acid (OA), and are thought to be derived from the starter molecule hexanoyl-CoA. The other group (PET) share a bibenzyl structure characteristic of the BBA. The latter group is thought to be derived from dihydrocinnamoyl-CoA or dihydrocoumaroyl-CoA, to generate BBAs.

Whereas some cannabinoids, e.g. THCA and CBDA, can be extracted from plants, in particular Cannabis sativa, there is an increasing interest in producing these molecules, and derivatives thereof by microbial fermentation. This will allow the study of their physiological effects, with the aim of identifying compounds with positive effects regarding human health. The many biological activities of cannabinoids has been recently reviewed, e.g. by Russo 2011 and Carvalho et al. 2017. Hence, due to the close structural relationship between cannabinoids and bibenzylic acids and derivatives, it is of major interest to study whether BBA derived molecules will have benefits and applications in the same or related pharmaceutical areas as for cannabinoids. Some indications of this are the fact that BBAs (aka amorfrutins) are already known to interact with the PPAR gamma receptor (Sauer et al. 2014), and that molecules like perrottetinene (see FIG. 3) are known to interact with the cannabinoid receptors (Chicca et al. 2019). Unfortunately, the natural occurrence of the BBA derived molecules is very limited and, as for cannabinoids, there is a serious unmet need of producing the BBA derived compounds by microbial fermentation. The current invention provides the framework and technology that can achieve this.

After the early discoveries of BBAs, also known as stilbene carboxylates (STC), in certain plants, it was hypothesised that these structures were the products, intermediates, or derailment products of a single polyketide Type III synthase (PKS). However, Shibuya et al. (2002) demonstrated that STC is not a direct intermediate in the pathway to stilbenes, and Eckermann et al. (2003) reported having identified an enzyme responsible for making the related dihydro-stilbene carboxylate (DSTC), in this case a 5-hydroxy lunularic acid. Hence, they named the enzyme stilbene carboxylate synthase (STCS). However, activity of STCS was only demonstrated in vitro with a purified, recombinant enzyme and with dihydro-coumaroyl-CoA as substrate and has never been confirmed in vivo. Later, Austin et al. (2004) briefly mentioned the possibility of an unknown, auxiliary protein being responsible for the aldol cyclization of STCs. However, the authors proposed that the STC is likely derived from a rearrangement of the coumaroyl-triacetic acid lactone (CTAL), a derailment product of STS and similar PKS enzymes. In 2012 Gagne and co-workers (Gagne et al. 2012) demonstrared that olivetolic acid is made by a PKS and a tetraketide cyclase (TKC). They identified the polyketide cyclase OAC (olivetolic acid cyclase) to be responsible for the Aldol C2-C7 cyclization of a tetraketide precursor, with retention of the carboxyl group, to form olivetolic acid. The production of olivetolic acid was proposed to proceed from a tetraketide-CoA, which is released by the PKS (renamed to TKS for tetraketide synthase). They suggested that there may be an overlooked class of polyketide cyclases, structurally related to DABB enzymes from bacteria and stress proteins in plants. However, no new such cyclases have been reported to date. In summary, Gagne et al. (2012) showed that some polyketides (PKs) are formed by the concerted action of a PKS and a cyclase. Despite speculations that other PKs could be formed by such a two-step pathway, they did not mention that this could be the case for STCs or DSTCs.

Mechanistic studies of OAC were done by Yang et al. 2016. They refer to OAC as the only known plant PK cyclase, and the only characterized DABB protein from plants. The crystal structure of OAC was presented and the authors stated that it has a unique active site, including a pentyl binding pocket, responsible for binding the linear pentyl-tetra-β-ketide-CoA, the natural product of the C. sativa TKS. A number of proteins with homology to OAC, e.g. from A. thaliana, did not catalyse the cyclization (as already shown by Gagne), and they concluded that this is due to differences in size and shape of the active site.

Another study was done by Taura et al. (2016) in which they demonstrated that orselinic acid can be produced by the PKS orselinic acid synthase (ORS) and OAC, in a reaction similar to what was shown for the PKS (TKS) and OAC in the production of olivetolic acid. This showed that, in addition to the natural pentyl-tetra-β-ketide-CoA, which is used for production of olivetolic acid, the OAC can accept other substrates, however in this case the much smaller methyl-tetra-β-ketide-CoA produced by ORS.

Most recently it was shown that various other short fatty acids, in addition to hexanoic acid, are accepted as substrates by the TKC (OAC) responsible for producing OA, and yields novel OA-like compounds (Luo et al. 2019)

In two recent publications (Weidner et al. 2012, de Groot et al. 2013) two bioactive BBA derived molecules were reported, amorfrutin A1 and amorfrutin B, respectively, which the authors refer to as dihydrostilbene carboxylates (FIG. 1). These molecules were shown to modulate the peroxisome proliferator-activated receptor PPARγ, a nuclear receptor which regulates lipid and glucose metabolism in humans. It was demonstrated that the amorfrutins improved insulin sensitivity and did not have the unwanted side effect of inducing weight gain, a feature exhibited by existing diabetes drugs like rosiglitazone. The authors suggested that amorfrutins may represent a new class of natural molecules with potential for treatment or prevention of type II diabetes and metabolic syndrome (Weidner et al. 2012, de Groot et al. 2013, Sauer 2014).

Despite being natural compounds, no larger scale production of specific amorfrutins or other BBA derivatives from plant extracts has been developed, and instead a chemical synthesis route was developed in order to test the two compounds (de Groot et al. 2013).

The biosynthetic pathway of THCA and CBDA, starting from hexanoyl-CoA, have been described (Schachtsiek et al. 2018) and starts with the precursor hexanoyl-CoA, which is extended by the Cannabis sativa tetraketide synthase (TKS), a type III polyketide synthase (PKS), with 3 units of malonyl-CoA into a tetraketide, which is subsequently cyclized into olivetolic acid (OA) by the C. sativa olivetolic acid cyclase (OAC). OAC is, to date, the only known polyketide cyclase (PKC). Similar biosynthetic pathways, depending on PKS enzymes, are well known from plants, such as those involved in the production of stilbenes and chalcones. These pathways start from phenylpropanoyl-CoA precursors, and are extended, as for OA, with 3 units of malonyl-CoA. However, unlike olivetolic acid, the stilbenes and chalcones are made by a single PKS enzyme, the stilbene synthase (STS) and the chalcone synthase (CHS), respectively, without the need for an accessory cyclase.

Nevertheless, it is known that these PKS enzymes, in particular of the STS type, also produces non-cyclized polyketides, either tri-ketides or tetraketides (Austin et al. 2004) which can spontaneously and non-enzymatically cyclize by lactonization or aldol cyclization. It is also known that PKS enzymes are able to accept phenylpropanoyl-CoA substrates in which the C2-C3 alkene bond has been reduced, e.g. by the yeast double bond reductase (DBR) ScTSC13 (Eichenberger et al. 2017). In that case, using CHS-type PKS enzymes, the production of dihydrochalcones, e.g. phloretin, was reported.

In 2012 the cyclase CsOAC was identified (Gagne 2012) and the authors reported that this cyclase did not need to physically interact with the PKS (in this case the CsTKS) in order to produce OA, and the authors could not detect any physical interaction. The study showed, however, that when CsOAC and CsTKS were assayed together in the same chamber, thus allowing physical interaction, the production of OA was considerably higher.

### SUMMARY OF THE INVENTION

Providing technical improvement, advantages and/or advancements over the background art the present invention provides solutions to technical problems identified in the art. In particular in view of the background art the present inventor has surprisingly found that the OAC is able to accept the much larger dihydro-cinnamoyl-tetra-β-ketide-CoA, and use this substrate to produce a BBA, i.e. dihydro stilbene carboxylate and that for studying the potential health benefits of these compounds, and for sustainable commercial production, a process involving fermentation from genetically modified microorganisms e.g. yeast will be attractive. It was further found by the present inventor that STS-type PKS enzymes are also able to use reduced phenylpropanoyl-CoA precursors as substrate. Together with 3 units of malonyl-CoA this would result in the formation of dihydro-tetraketide intermediates, and it has been found that the further co-expression of a PKC, leads to formation of bibenzylic acids (BBA) (FIG. 1 and FIG. 2), which are structurally similar to OA. The present inventor has further found that the production of BBA depends on the release of free, non-cyclized tetraketides from the PKS. In the case of STS-type PKS enzymes, this was reflected in the ratio between free tetraketide and the cyclized bibenzyl, e.g. dihydropinosylvin (FIG. 2). The ratio is determined by the substrate specificity and the efficiency of the C2-C7 aldol condensation. Different PKS enzymes therefore exhibit different ratios and overall efficiency, and the PKS can be optimised for BBA production, e.g. by mutagenesis, in particular by mutations interfering with the aldol cyclization. Similarly, other PKS enzymes, other than STS-type, can be mutated, adapted, or engineered to release the free tetraketide, which can then be cyclized by OAC—one example being the alfalfa CHS mutant T197L (Austin 2004).

Accordingly, through the discovery that (i) the polyketide synthases (PKS), preferably of the STS type, when using a reduced dihydro-phenylpropanoid-CoA precursor, release substantial amounts of free tetraketide, as compared to the normal production of flavonoid compounds such as dihydropinosylvin, in the case of CHS, or stilbenoid compounds such as dihydroresveratrol, in the case of STS, and that (ii) the free tetraketides produced by the PKS are substrates of cyclases, such as the OAC, despite the fact that these phenylpropanoid derived tetraketides are much more bulky than other known substrates of polyketide cyclases, a first aspect of the present invention provides a genetically modified microbial host cell capable of producing bibenzylic acids or derivatives thereof wherein the genetically modified host cell expresses:
a) one or more genes encoding a polyketide synthase (PKS);
b) one or more genes encoding a polyketide cyclase (PKC);
c) one or more genes genes encoding a double bond reductase (DBR); and
   and one or more genes encoding
d) a tyrosine ammonia lyase polypeptide;
e) a phenylalanine ammonia lyase polypeptide;
f) a cinnamate 4-hydroxylase polypeptide;
g) a cytochrome p450 reductase polypeptide;
h) a 4-coumarate-CoA ligase polypeptide; and/or
i) a chalcone isomerase type III or IV polypeptide;
   wherein the at least one gene is heterologous to the host cell.

In further aspects the invention provides a cell culture, comprising the cell of the invention and a growth medium as well as a method of producing the BBA or a derivative thereof, comprising:
a) culturing the cell culture of the invention at conditions allowing the host cells to produce the BBA or a derivative thereof; and
b) optionally recovering and/or isolating the BBA or a derivative thereof.

In a still further aspect, the invention provides a fermentation composition comprising the cell culture of the invention and the BBA or a derivative thereof, optionally in the form of a dimer, as well as compositions comprising the fermentation composition of the invention and one or more carriers, agents, adjuvants, additives and/or excipients; and the use of said composition of the invention for use as a medicament. This aspect also includes a method for treating a disease in a mammal, comprising administering a therapeutically effective amount of the composition of the invention to the mammal.

### DESCRIPTION OF DRAWINGS AND FIGURES

The features and advantages of the present invention is readily apparent to those skilled in the art by the below detailed description of embodiments and examples of the invention with reference to the figures and drawings included herein where:

FIG. 1: Production of Bibenzylic Acid (BBA) in Yeast. In yeast, bibenzylic acid can be prepared by a heterologous, biosynthetic pathway starting from the aromatic amino acids phenylalanine (shown) or tyrosine by introduction of the plant genes PAL, 4CL, DBR, PKS and TKC.

FIG. 2: Mechanism for production of tetraketide and BBA. PKS enzymes like CHS and STS use (dihydro-)phenylpropanoyl-CoA and 3 molecules of malonyl-CoA to synthesize a tetraketide. The fate of the polyketide depends on the type of enzyme but can also be released as free CoA-linked tetraketide before cyclization, allowing this intermediate to be cyclised by a TKC enzyme, e.g. ScOAC, to form a BBA. Release of the tetraketide is shown to be common among STS-type enzymes.

FIG. 3: THC vs PET biosynthesis. Illustration of the similarity between the biosynthetic pathways of the various compounds derived from either olivetolic acid or from a bibenzylic acid.

FIG. 4: Illustration of the biosynthesis of THC and CBD from Cannabis sativa, as compared to the structurally similar bibenzylic cannabinods PET and PTD.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

### Definitions

The term “PKS” or “polyketide synthase” or “polyketide type III synthase” as used herein refers to an enzyme catalyzing the extension of a CoA-activated substrate with one or more malonyl-CoA units. Chalcone synthase or CHS is one example of a type 3 polyketide synthase enzyme capable of synthesizing a chalcone by condensing 3 molecules of malonyl-CoA with a phenylpropanoyl-CoA (aka (hydroxy)-cinnamoyl-CoA), such as a naringenin chalcone from one molecule of p-coumaroyl-CoA and three molecules of malonyl-CoA. Another example of a PKS is orselinic acid synthase (ORS) capable of catalyzing conversion of acetyl-CoA plus three molecules of malonyl-COA into orselinic acid. Another example of a PKS is tetraketide synthase or TKS capable of catalyzing conversion of hexanoyl-CoA plus three molecules of malonyl-CoA into a tetraketide, the normal substrate of CsOAC. Another example of a PKS is stilbene synthase or STS, a type 3 polyketide synthase enzyme capable of catalyzing the formation of a stilbene or dihydrostilbene from one molecule of (dihydro-)cinnamoyl-CoA or (dihydro-) p-coumaroyl-CoA and three molecules of malonyl-CoA.

The term “CHIL” as used herein refers to chalcone isomerase-like protein, a polypeptide also known as the non-catalytic CHI types III and IV.

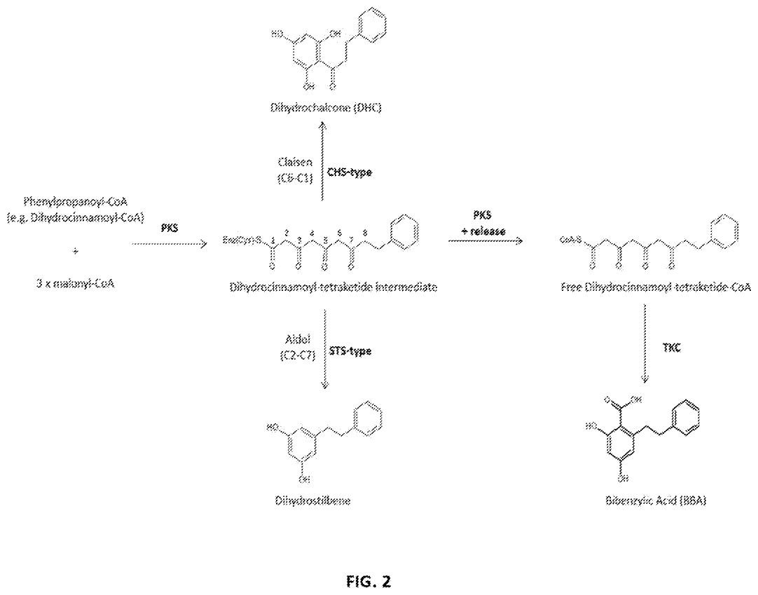

The term “BBA” or “Bibenzylic acid” or derivatives thereof as used herein refers to a compound in which two phenyl groups are linked via a 2-carbon bridge, and one phenyl group carries a carboxyl group, such a compound also known as a stilbene carboxylate (STC) or a dihydro-stilbene carboxylate (DSTC) (FIG. 2)

The term “STC” or “stilbene carboxylate” as used herein refers to a stilbene in which one of the phenyl groups carries a carboxyl group. The carbons linking the phenyl groups are themselves connected by a double bond, unlike the DSTC in which they are connected by a single bond.

The term “STCS” or “stilbene carboxylate synthase” as used herein refers to an enzyme catalysing conversion of a (dihydro-)-(hydroxy-)-cinnamoyl-CoA starter molecule plus three molecules of malonyl-CoA into an STC or an DSTC in the genetically modified microorganism.

The term “DSTC” or “dihydro-stilbene carboxylate” as used herein refers to a BBA in which the carbons linking the two phenyl groups are connected via a single bond

The term “coumaroyl-triacetic acid lactone” (CTAL) as used herein refers to a compound formed by spontaneous lactonization of a free p-coumaroyl-tetraketide (aka p-coumaroyl-triacetic acid) released from a PKS type III enzyme such as CHS, TKS, or STS

The term “PAL” as used herein refers to phenylalanine ammonia lyase, an enzyme catalyzing conversion of phenylalanine to cinnamic acid.

The term “4CL” as used herein refers to 4-coumarate-CoA-ligase, an enzyme catalyzing conversion of the ligation of CoA to various phenylpropanoic acids.

The term “tetraketide cyclase” or “TKC” or “polyketide cyclase” or “PKC” as used herein refers to an enzyme catalyzing conversion of a free tetraketide to a phenolic acid, such as orselinic acid, oliveetolic acid, or bibenzylic acid. These terms are used interchangeably. One example of a tetraketide cyclase is olivetolic acid cyclase or OAC.

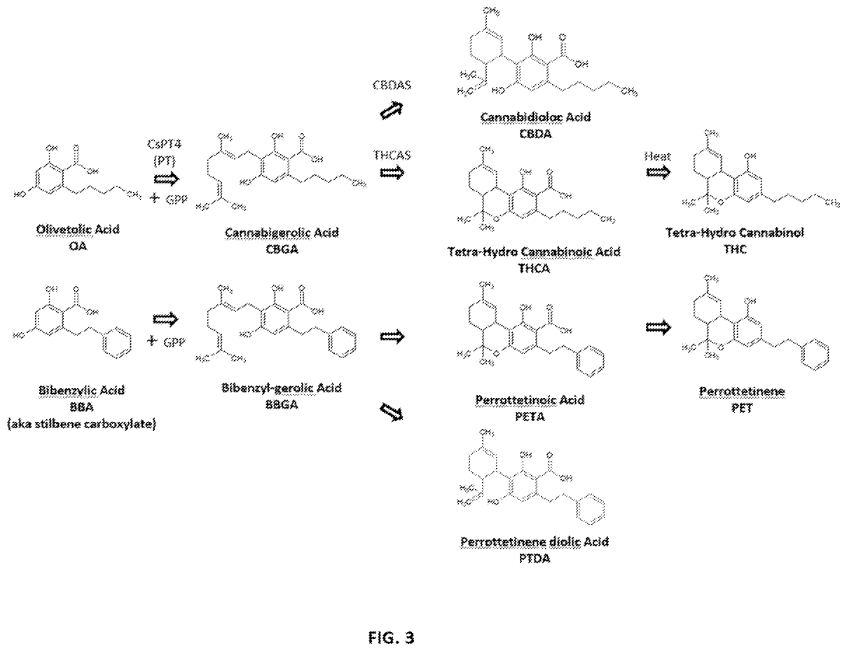

The term “OA as used herein refers to olivetolic acid (FIG. 3).

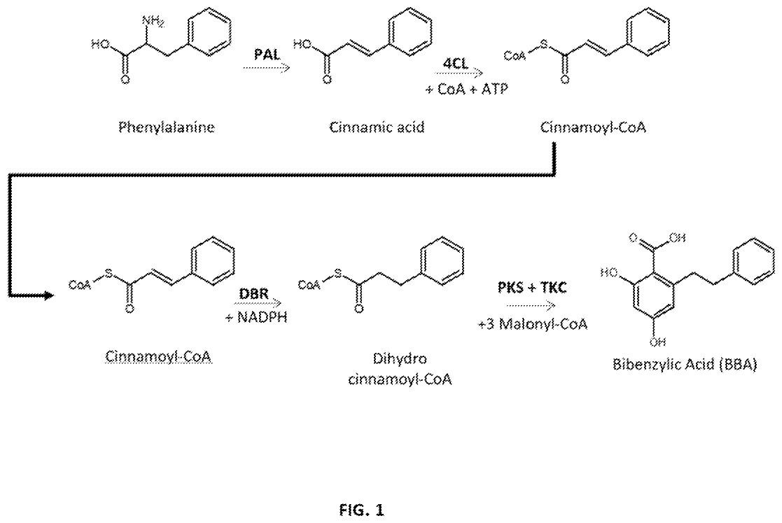

The term “double bond reductase” or “DBR” refers to an enzyme catalyzing reduction of the C2-C3 double bond of one or more substrates selected from cinnamoyl-CoA, p-Coumaroyl-CoA, Caffeoyl-CoA, Feruoyl-CoA into the respective dihydrocinnamoyl-CoA, p-dihydrocoumaroyl-CoA, dihydrocaffeoyl-CoA and dihydroferuloyl-CoA (FIG. 1).

The term “cinnamate 4-hydroxylase” or “C4H” as used herein refers to a CYP450 trans-cinnamate 4-monooxygenase enzyme also known as cinnamate 4-hydroxylase, catalyzing conversion of cinnamic acid to p-coumaric acid.

The term “CYP450 as used herein” refers to an enzyme of the Cytochrome P450 family, catalysing oxidation of a range of substrates. Upon acting on a substrate, CYP450 must be reduced by its cognate reductase (CPR) to regain catalytic capacity.

The term “cytochrome p450 reductase” or “CPR” as used herein refers to an enzyme catalyzing the reduction of CYP450 enzymes.

The term “tyrosine ammonia lyase” or “TAL” as used herein refers to an enzyme catalyzing conversion of tyrosine to p-coumaric acid.

The term “4-coumarate-CoA ligase” or “4CL” as used herein refers to an enzyme catalyzing conversion of the ligation of CoA to various phenylpropanoic acids.

The term “chalcone isomerase type” or “CHI” as used herein refers to an enzyme catalysing stereospecifical isomerization of a chalcone to a (2S)-flavanone.

The terms “heterologous” or “recombinant” or “genetically modified” and their grammatical equivalents as used herein interchangeably refers to entities “derived from a different species or cell”. For example, a heterologous or recombinant polynucleotide gene is a gene in a host cell not naturally containing that gene, i.e. the gene is from a different species or cell type than the host cell. The terms as used herein about host cells refers to host cells comprising and expressing heterologous or recombinant polynucleotide genes.

The term “pathway” or “biosynthetic pathway” or “metabolic pathway” as used herein is intended to mean an enzyme acting in a live cell to convert a chemical substrate into a chemical product. A pathway may include one enzyme or multiple enzymes acting in sequence. A pathway including only one enzyme may also herein be referred to as “bioconversion” in particular relevant for embodiments where the cell of the invention is fed with a precursor or substrate to be converted by the enzyme into a desired product. Enzymes are characterized by having catalytic activity, which can change the chemical structure of the substrate(s). An enzyme may have more than one substrate and produce more than one product. The enzyme may also depend on cofactors, which can be inorganic chemical compounds or organic compounds (co-factor and/or co-enzymes or non-catalytic polypeptides). The NADPH-dependent cytochrome P450 reductase (CPR) is an electron donor to cytochromes P450 (CYPs). CPR shuttles electrons from NADPH through the Flavin Adenine Dinucleotide (FAD) and Flavin Mononucleotide (FMN) coenzymes into the iron of the prosthetic heme-group of the CYP. The term “operative biosynthetic metabolic pathway” refers to a metabolic pathway that occurs in a live recombinant host, as described herein.

The term “in vivo”, as used herein refers to within a living cell or organism, including, for example animal, a plant or a microorganism.

The term “in vitro”, as used herein refers to outside a living cell or organism, including, without limitation, for example, in a microwell plate, a tube, a flask, a beaker, a tank, a reactor and the like.

The term “substrate” or “precursor”, as used herein refers to any compound that can be converted into a different compound. For clarity, substrates and/or precursors include both compounds generated in situ by an enzymatic reaction in a cell or exogenously provided compounds, such as exogenously provided organic molecules which the host cell can metabolize into a desired compound.

Term “endogenous” or “native” as used herein refers to a gene or a polypepetide in a host cell which originates from the same host cell.

The term “deletion” as used herein refers to manipulation of a gene so that it is no longer expressed in a host cell.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that the expression of the gene is reduced as compared to expression without the manipulation.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but is not limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “% identity” is used herein about the relatedness between two amino acid sequences or between two nucleotide sequences. “% identity” when used herein about amino acid or nucleotide sequences refers to the degree of identity in percent between two amino acid sequences obtained when using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$\frac{i d e n tical amino acid residues}{L ength of alignment - total number of gaps in alignment} \times 100$$

“% identity” when used herein about nucleotide sequences refers to the degree of identity in percent between two nucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

$$\frac{identical deoxyribonucleotides}{Length of alignment - total number of gaps in alignment} \times 100$$

The protein sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:

Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins.

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity. Alternatively, % identity for any candidate nucleic acid or amino acid sequence relative to a reference sequence can be determined as follows. A reference sequence (e.g., a nucleic acid sequence or an amino acid sequence described herein) is aligned to one or more candidate sequences using the computer program Clustal Omega (version 1.2.1, default parameters), which allows alignments of nucleic acid or polypeptide sequences to be carried out across their entire length (global alignment). Chenna et al., 2003, Nucleic Acids Res. 31(13):3497-500. Clustal Omega calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: % age; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method:% age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The Clustal Omega output is a sequence alignment that reflects the relationship between sequences. Clustal Omega can be run, for example, at the Baylor College of Medicine Search Launcher site on the World Wide Web (searchlauncher.bcm.tmc.edu/multi-align/multi-align.html) and at the European Bioinformatics Institute site at http://www.ebi.ac.uk/Tools/msa/clustalo/. To determine a % identity of a candidate nucleic acid or amino acid sequence to a reference sequence, the sequences are aligned using Clustal Omega, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” or “promoter” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression” includes any step involved in the production of a polypeptide including, but not limited to, transcription, post-transcriptional modification, translation, post-translational modification, and secretion.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “host cell” refers to any cell type that is susceptible to transformation, transfection, transduction, or the like with a nucleic acid construct or expression vector comprising a polynucleotide of the present invention. Host cell encompasses any progeny of a parent cell that is not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “operably linked” refers to a configuration in which a control sequence is placed at an appropriate position relative to the coding polynucleotide such that the control sequence directs expression of the coding polynucleotide.

The terms “nucleotide sequence and “polynucleotide” are used herein interchangeably.

The term “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The term “express” as used herein, refers to a gene which is transcribed and translated in a cell to produce a peptide or polypeptide.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the itemed invention or to imply that certain features are critical, essential, or even important to the structure or function of the itemed invention. Rather, these terms are merely intended to highlight alternative or additional features that can or cannot be utilized in a particular embodiment of the present invention.

The term “cell culture” as used herein refers to a culture medium comprising a plurality of host cells of the invention. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

### Genetically Modified Microorganisms

The genetically modified host cell of the invention produces bibenzylic acids or a derivative thereof due to the operation of a metabolic pathway expressing:
a) one or more genes encoding a polyketide synthase (PKS), optionally a stilbene type PKS;
b) one or more genes encoding a polyketide cyclase (PKC); and
c) one or more genes genes encoding a double bond reductase (DBR);
   and one or more genes encoding polypeptides selected from
d) a tyrosine ammonia lyase polypeptide (TAL);
e) a phenylalanine ammonia lyase polypeptide (PAL);
f) a cinnamate 4-hydroxylase polypeptide (C4H);
g) a cytochrome p450 reductase polypeptide (CPR);
h) a 4-coumarate-CoA ligase polypeptide (4CL); and/or
i) a chalcone isomerase-like type III or IV polypeptide (CHIL);
   wherein the at least one of the genes is heterologous to the host cell.

In some embodiments the double bond reductase is a native enoyl-reductase, and optionally it is overexpressed at least two-fold compared to the native expression level. In other embodiments the double bond reductase is a heterologous reductase capable of reducing the alkene C2-C3 double bond of a phenylpropanoid or phenylpropanoyl-CoA precursor. The double bond reductase of the invention can be one which comprises an amino acid sequence which is at least 70%, such as at least 80%, such as at least 90%, such as at least 95%, such as at least 96%, such as at least 97%, such as at least 98%, such as at least 99%, such as 100% identical to the double bond reductase comprised in SEQ ID NO: 3.

In further embodiments a polyketide synthase which produces a linear tetraketide, and/or a free activated linear tetraketide-CoA, optionally from a dihydrophenylpropanoid, is selected. Such polyketide synthases include polyketide synthases which comprises an amino acid sequence which is at least 70%, such as at least 80%, such as at least 90%, such as at least 95%, such as at least 96%, such as at least 97%, such as at least 98%, such as at least 99%, such as 100% identical to the stilbene type polyketide synthases comprised in SEQ ID NO: 4, 5, 6, 7, 8, 9, or 10. In a particular embodiment the polyketide synthase has little or no activity towards C2-C7 aldol condensation on a tetraketide substrate, so that or example less than 70% of tetraketide substrate conversion produces a C2-C7 aldol condensation, such as less than 50%, such as less than 30%, such as less than 10% of, such as less that 5%. Reduction or elimination of activity towards C2-C7 aldol condensation can optionally be optimized by mutating the polyketide synthase to achieve an inactivating mutation. The active sites of both CHS and STS type enzymes have been elucidated, including specific residues involved in the so-called aldol switch in STS (Austin et al. 2004). Targeting these residues can disrupt the cyclization, as has been shown for the CHS T197L mutation, which releases free tetraketides.

In some embodiments, the polyketide cyclase comprises an amino acid sequence which is at least 70%, such as at least 80%, such as at least 90%, such as at least 95%, such as at least 96%, such as at least 97%, such as at least 98%, such as at least 99%, such as 100% identical to the polyketide cyclase comprised in SEQ ID NO: 11. More specifically the polyketide cyclase is the olivetolic acid cyclase comprised in SEQ ID NO. 11.

The tyrosine ammonia lyase is suitably one which comprises an amino acid sequence which is at least 70%, such as at least 80%, such as at least 90%, such as at least 95%, such as at least 96%, such as at least 97%, such as at least 98%, such as at least 99%, such as 100% identical to the tyrosine ammonia lyase comprised in SEQ ID NO: 25.

The phenylalanine ammonia lyase is suitably one which comprises an amino acid sequence which is at least 70%, such as at least 80%, such as at least 90%, such as at least 95%, such as at least 96%, such as at least 97%, such as at least 98%, such as at least 99%, such as 100% identical to the phenylalanine ammonia lyase comprised in SEQ ID NO: 1.

The cinnamate 4-hydroxylase is suitably one which comprises an amino acid sequence which is at least 70%, such as at least 80%, such as at least 90%, such as at least 95%, such as at least 96%, such as at least 97%, such as at least 98%, such as at least 99%, such as 100% identical to the cinnamate 4-hydroxylase comprised in SEQ ID NO: 26.

The cytochrome p450 reductase is suitably one which comprises amino acid sequence which is at least 70%, such as at least 80%, such as at least 90%, such as at least 95%, such as at least 96%, such as at least 97%, such as at least 98%, such as at least 99%, such as 100% identical to the cytochrome p450 reductase comprised in SEQ ID NO: 27.

The BBAs can be further modified, e.g. by prenylation, to eventually yield derivatives, such as perrottetinoic acid (PETA, FIG. 3) and its decarboxylate perrottetinene (PET). Surprisingly, this can be achieved by expressing enzymes from the cannabinoid biosynthetic pathway, which as demonstrated herein have very relaxed substrate specificity. In summary, this opens up the area of BBAs and derivatives for microbial production, and eventually their application in the human health sector. Accordingly, in some embodiments the cell of the invention further expresses a gene encoding a prenyl transferase, particularly a prenyl transferase which is a geranyl transferase. The prenyl transferase preferably transfers a prenyl group to a bibenzylic acid. The prenyl transferase is suitably one which comprises an amino acid sequence which is at least 70%, such as at least 80%, such as at least 90%, such as at least 95%, such as at least 96%, such as at least 97%, such as at least 98%, such as at least 99%, such as 100% identical to the prenyl transferase comprised in SEQ ID NO: 15.

In further embodiments the cell of the invention further expresses a gene encoding a non-catalytic chalcone isomerase like polypeptide (CHIL), enhancing the production of BBA or derivatives thereof. The non-catalytic chalcone isomerase like polypeptide is suitably one which comprises an amino acid sequence which is at least 65%, such as at least 70%, such as at least 80%, such as at least 90%, such as at least 95%, such as at least 96%, such as at least 97%, such as at least 98%, such as at least 99%, such as 100% identical to the non-catalytic chalcone isomerase like polypeptide comprised in SEQ ID NO: 23 or 24. Moreover, in one embodiment of the invention the OAC is replaced by a non-catalytic chalcone isomerase like (preferably type 4) protein, which via physical interaction with the PKS enzyme controls the reaction of the latter, to cyclize the formed tetraketide with retention of the C1 carboxy group.

The bibenzylic acid (BBA) of the invention is particularly BBA's as defined by formula (I):

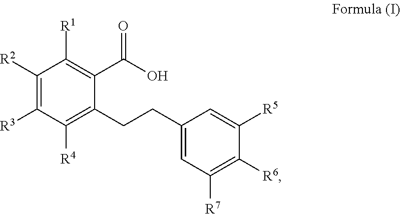

wherein R1-R7 can be either —H, —OH, —OCH3, —COOH, an acyl group, or defined by formula (II):

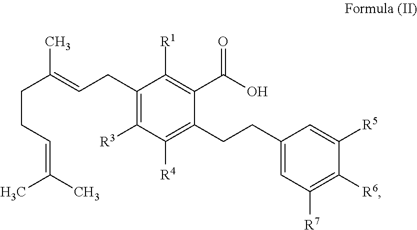

wherein R1, and R3-R7 can be either —H, —OH, —OCH3, —COOH, an acyl group, or a prenyl group, or defined by Formula (III):

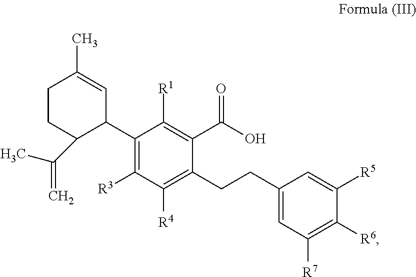

wherein R1, and R3-R7 can be either —H, —OH, —OCH3, —COOH, an acyl group, or a prenyl group, or defined by Formula (IV):

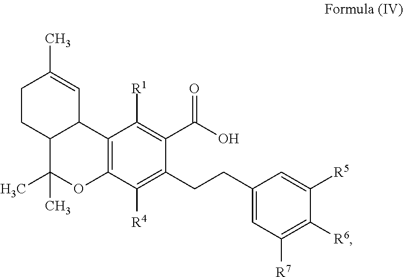

wherein R1, and R4-R7 can be either —H, —OH, —OCH3, —COOH, an acyl group, or a prenyl group.

Accordingly, the cell of the invention expresses the enzymes to synthesize the phenylpropanoyl-CoA precursor, a double bond reductase such as ScTSC13 to reduce the C2-C3 double bond of said precursor, a PKS enzyme, and a CHIL type 4 protein, capable of interacting with the PKS, resulting in the formation of a BBA as defined by formula (I).

Any of the groups R1-R7 can subsequently be rearranged or be further modified, spontaneously, biosynthetically, or synthetically. Accordingly, in further embodiments the cell of the invention expresses a gene encoding a synthase converting a compound of Formula (II) into a compound of formula (III). In particular such synthase comprises an amino acid sequence which is at least 70%, such as at least 80%, such as at least 90%, such as at least 95%, such as at least 96%, such as at least 97%, such as at least 98%, such as at least 99%, such as 100% identical to the synthase comprised in SEQ ID NO: 22.

In further embodiments, the cell of the invention may express a gene encoding a synthase converting a compound of Formula (II) into a compound as defined by Formula (IV). In particular such synthase comprises an amino acid sequence which is at least 70%, such as at least 80%, such as at least 90%, such as at least 95%, such as at least 96%, such as at least 97%, such as at least 98%, such as at least 99%, such as 100% identical to the synthase comprised in SEQ ID NO: 21.

It is also contemplated that the CHIL type 4 interacts with the PKS to enhance the production of free polyketide. The PKS in this embodiment can be any PKS, such as an STS or mutated CHS, capable of releasing the free tetraketide. Hence, in another embodiment the cell of the invention expresses the enzymes to synthesize the phenylpropanoyl-CoA precursor, a double bond reductase such as ScTSC13 to reduce the C2-C3 double bond of said precursor, a PKS enzyme, a CHIL type 4 protein, capable of interacting with the PKS, resulting in the increased formation of a free tetraketide, and a PKC such as CsOAC, capable of cyclizing said tetraketide to form a BBA as defined by Formula (I), above.

The co-expression of a CHIL type 4 protein, capable of positively interacting with the relevant PKS, has positive implications for the production of dihydrochalcones, such as those described by Eichenberger (2017). Further, if the PKS, such as the one described by Pluskal (2019), is efficiently releasing triketides or dihydro-triketides, the co-expression of a CHIL type 4 protein has a beneficial effect on the production of kavalactones including bisnoryangonin, yangonin, or its corresponding dihydro-versions.

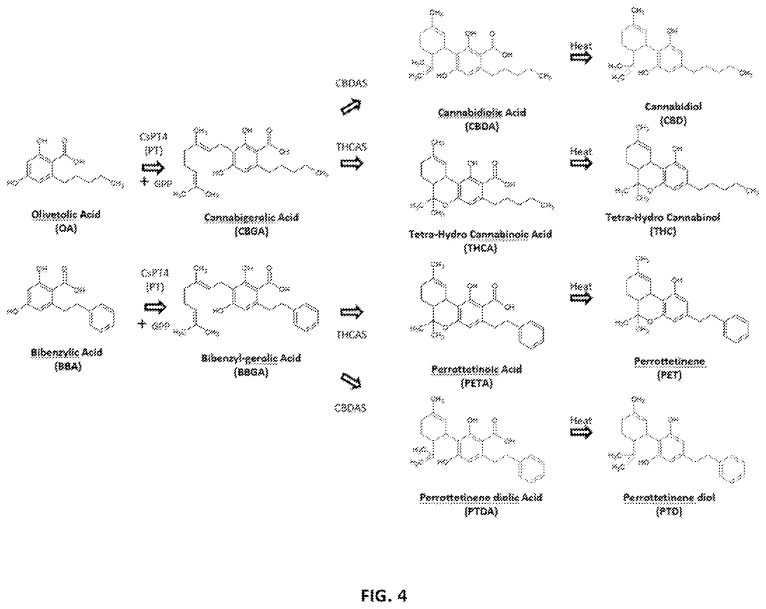

The full biosynthetic pathway from OA to some of the most important cannabinoids THCA (tetrahydrocannabinolic acid) and CBDA (cannabidiolic acid) has recently been expressed in a microbial host (Saccharomyces cerevisiae) (Luo et al. 2019). The pathway expresses the prenyl transferase (CsPT4) from Cannabis sativa that transfers a geranyl group to OA to produce cannabigerolic acid (CBGA), followed by the cyclization of the geranyl side group by two different synthases to form either THCA (by THCA synthase) or CBDA (by CBDA synthases). Heat treatment is known to convert THCA and CBDA into THC (tetrahydrocannabinol) and CBD (cannabidiol), respectively (FIG. 4). The present inventor now found that a prenyl transferase (CsPT4) and a THCA synthase (THCAS) from Cannabis sativa converted BBA into the prenylated bibenzylgerolic acid (BBGA) and further into PETA, a (FIG. 4). When the CBDA synthase (CBDAS) is introduced, together with the CsPT4, into the yeast strain producing BBA, the formation of perrottetinene diolic acid (PTDA) is detected (FIG. 4).

In certain embodiments it is advantageous to change the cellular localization of proteins or enzymes of the invention. For example, the expression of THCA synthase (THCAS) and CBDA synthase (CBDAS) may advantageously be expressed in the vacuole of the host cell. This can be achieved by changing the localization signal of the native protein with one that directs the transport to the vacuole. An example of this is described by Zirpel et al. 2015, in which the use of a vacuolar localization signal (MIFDGTTMSIAIGLLSTLGIGAEA) from the protease proteinase A (Acc. No. F2QUG8) allows expression of THCAS in a yeast host cell. Accordingly, the THCAS and CBDAS, as used in the current invention, were both synthesized in such a way as to remove the N-terminal signal sequence of 28 amino acids, which were then replaced by the vacuolar N-terminal signal, comprising 24 amino acids, of the proteinase A (Zirpel et al. 2015; Lou et al. 2019).

Similarly, certain embodiments may benefit from relocalization of prenyltransferases. Hence, the cellular localization of prenyltransferases, used for the current invention, were N-terminally truncated to remove the plastid targeting signal. Amino acid sequences of full-length proteins were aligned, and signal sequences were putatively identified at the N-terminal end. Synthetic genes, encoding these prenyltransferases, were then designed and synthesized, excluding between 62 and 88 amino acids at the N-terminal end, resulting in the truncated prenyltransferases (SEQ ID NOS: 13-20) used for the current invention.

In certain embodiments it is advantageous to co-express a non-catalytic chalcone isomerase type III or IV protein (CHIL) in the host cell producing bibenzylic acids, bibenzylgerolic acid, or derivatives thereof. It appears that such CHIL proteins can promote polyketide formation. Alternatively, CHIL proteins may stabilize intermediates or end products by directly binding to them. The current invention demonstrates increased production of THCA when HICHIL2 (SEQ ID NO: 24) is co-expressed together with the full length THCA biosynthetic pathway. It is contemplated that different CHIL proteins have different function as accessory protein for various reactions during polyketide biosynthesis, and that specific CHILs can be found to support specific functions. In certain embodiments it is advantageous to engineer the host cell to produce more of the precursors used for the desired product. For example, it will be advantageous for production of bibenzylic acid (BBA) to optimize the host for production of the natural precursor molecules such as the aromatic amino acids phenylalanine and/or tyrosine, as well as the precursors acetyl-CoA and malonyl-CoA.

The cell of the invention is suitably selected from the genera consisting of Saccharomyces, Schizosaccharomyces, Yarrowia, Candida, Ashbya, Cyberlindnera, Pichia, Kluyveromyces, Hansenula, Arxula, and Xanthophyllomyces, optionally from the species Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, and Candida albicans.

### Cultures

Further provided for herein are cell cultures comprising the genetically modified host cells of the invention and a growth medium. Suitable growth mediums for relevant prokaryotic or eukaryotic host cells are videly known in the art.

### Methods of Producing Compounds of the Invention.

The invention also provides a method for producing BBA or derivatives thereof comprising
a) culturing the cell culture of the invention at conditions allowing the host cells to produce the BBA or derivatives thereof; and
b) optionally recovering and/or isolating the BBA or derivatives thereof.

The cell culture can be cultivated in a nutrient medium and at conditions suitable for production of the BBA or derivatives thereof of the invention and/or for propagating cell count using methods known in the art. For example, the culture may be cultivated by shake flask cultivation, or small-scale or large-scale fermentation (including continuous, batch, fed-batch, or solid-state fermentations) in laboratory or industrial fermenters in a suitable medium and under conditions allowing the host cells to grow and/or propagate, optionally to be recovered and/or isolated. The cultivation can take place in a suitable nutrient medium comprising carbon and nitrogen sources and inorganic salts, using procedures known in the art. Suitable media are available from commercial suppliers or may be prepared according to published recipes (e.g. from catalogues of the American Type Culture Collection). The selection of the appropriate medium may be based on the choice of host cell and/or based on the regulatory requirements for the host cell. Such media are available in the art. The medium may, if desired, contain additional components favoring the transformed expression hosts over other potentially contaminating microorganisms. Accordingly, in an embodiment a suitable nutrient medium can include one or more of (i) trace metals; (ii) vitamins; (iii) salts (such as salts of phosphate, magnesium, potassium, zinc, iron); (iv) nitrogen sources (such as YNB, ammonium sulfate, urea, yeast extracts, ammonium nitrate, ammonium chloride, malt extract, peptone and/or amino acids); (v) carbon source (such as dextrose, sucrose, glycerol, glucose, maltose, molasses, starch, cellulose, xylan, pectin, lignocellolytic biomass hydrolysate, and/or acetate); (vi) nucleobases; (vii) aminoglycosides; and/or (viii) antibiotics (such as G418 and hygromycin B).

The cultivation of the host cell may be performed over a period of from about 0.5 to about 30 days. The cultivation process may be a batch process, continuous or fed-batch process, suitably performed at a temperature in the range of 0 to 100° C. or 0 to 80° C., for example, from about 0° C. to about 50° C. and/or at a pH, for example, from about 2 to about 10. Preferred fermentation conditions are a temperature in the range of from about 25° C. to about 55° C. and at a pH of from about 3 to about 9. The appropriate conditions are usually selected based on the choice of host cell. Accordingly, in some embodiments the method of the invention comprising one or more elements selected from:
a) culturing the cell culture under aerobic or anaerobic conditions
b) cultivating the host cells under mixing;
c) cultivating the host cells at a temperature of between 25° C. to 50° C.;
d) cultivating the host cells at a pH of between 3-9; and
e) cultivating the host cells for between 10 hours to 120 days.

The cell culture of the invention may be recovered and or isolated using methods known in the art. For example, the cells or compound(s) may be recovered from the nutrient medium by conventional procedures including, but not limited to, centrifugation, filtration, spray-drying, or lyophilization. In a particular embodiment the method includes a recovery and/or isolation step comprising separating a liquid phase of the cell culture from a solid phase of the cell culture to obtain a supernatant comprising the BBA or derivatives thereof and subjecting the supernatant to one or more steps selected from:
a) contacting the supernatant with one or more adsorbent resins in order to obtain at least a portion of the produced BBA or derivatives thereof, then optionally recovering the BBA or derivatives thereof from the resin in a concentrated solution prior to isolation of the BBA or derivatives thereof by crystallisation or solvent evaporation;
b) contacting the supernatant with one or more ion exchange or reversed-phase chromatography columns in order to obtain at least a portion of the BBA or derivatives thereof, then optionally recovering the BBA or derivatives thereof from the resin in a concentrated solution prior to isolation of the BBA or derivatives thereof by crystallisation or solvent evaporation;
c) extracting the BBA or derivatives thereof from the supernatant, such as by liquid-liquid extraction into an immiscible solvent, then optionally isolating the BBA or derivatives thereof by crystallisation or solvent evaporation; and
   thereby recovering and/or isolating the BBA or derivatives thereof.

The method of the invention may further comprise one or more steps of mixing the BBA or derivatives thereof with one or more carriers, agents, adjuvants, additives and/or excipients, optionally pharmaceutical grade carriers, agents, adjuvants, additives and/or excipients.

The method of the invention may further comprise one or more in vitro steps in the process of producing the BBA or derivatives thereof. It may also comprise one or more in vivo steps performed in another cell than the host cell of the invention. Accordingly, in one embodiment the method of the invention further comprises feeding one or more exogenous BBA precursors to the host cell culture.

### Fermentation Composition

The invention also provides a fermentation composition comprising the cell culture of the invention and the BBA or derivatives thereof—either comprised in the cells or in the medium. In the fermentation composition the genetically modified host cells may be wholly or partially lysed and/or disintegrated. In some embodiments at least 50%, such as at least 75%, such as at least 95%, such as at least 99% of the genetically modified host cells in the fermentation composition are lysed and/or disintegrated. Further, in the fermentation composition of the invention at least 50%, such as at least 75%, such as at least 95%, such as at least 99% of solid cellular material may have been separated and/or removed from a liquid phase of the fermentation composition.

Moreover, in addition to BBA or derivatives thereof the fermentation composition of the invention may comprise one or more compounds selected from precursor or products of the pathway producing the BBA or derivatives thereof, trace metals, vitamins, salts, yeast nitrogen base, carbon source, YNB, and/or amino acids of the fermentation. In particular the fermentation composition of the invention comprise a concentration of BBA or derivatives thereof of at least 1 mg/kg composition, such as at least 5 mg/kg, such as at least 10 mg/kg, such as at least 20 mg/kg, such as at least 50 mg/kg, such as at least 100 mg/kg, such as at least 500 mg/kg, such as at least 1000 mg/kg, such as at least 5000 mg/kg, such as at least 10000 mg/kg, such as at least 50000 mg/kg.

Suitable supplemental nutrients can include one or more of (i) trace metals; (ii) vitamins; (iii) salts (such as salts of phosphate, magnesium, potassium, zinc, iron); (iv) nitrogen sources (such as YNB, ammonium sulfate, urea, yeast extracts, ammonium nitrate, ammonium chloride, malt extract, peptone and/or amino acids); (v) carbon source (such as dextrose, sucrose, glycerol, glucose, maltose, molasses, starch, cellulose, xylan, pectin, lignocellolytic biomass hydrolysate, and/or acetate); (vi) nucleobases; (vii) aminoglycosides; and/or (viii) antibiotics (such as G418 and hygromycin B).

### Compositions and Use

The invention also provides a composition comprising the fermentation composition of the invention and one or more carriers, agents, adjuvants, additives and/or excipients. Suitable carriers, agents, adjuvants, additives and/or excipients includes formulation additives, stabilising agent, fillers and the like. The composition and the one or more carriers, agents, adjuvants, additives and/or excipients can suitably be formulated into in a dry solid form e.g by using methods known in the art, such as spray drying, spray cooling, lyophilization, flash freezing, granulation, microgranulation, encapsulation or microencapsulation. The composition and the one or more carriers, agents, adjuvants, additives and/or excipients can also be formulated into a liquid stabilized form using methods known in the art, such as adding to the fermentation composition one or more stabilizers such as sugars and/or polyols (e.g. sugar alcohols) and/or organic acids (e.g. lactic acid).

The composition of the invention may be further refined into a dietary supplement, a cosmetic, a food preparation, a feed preparation and/or an analytical or diagnostic reagent optionally using one or more steps of the methods described herein for producing the BBA or derivatives thereof. In one embodiment, BBA or derivative thereof and/or the composition comprising it can be used as a signal modulator of the cannabinoid receptor 1 (CB1), the cannabinoid receptor 2 (CB2) and/or the PPARgamma receptor.

### REFERENCES

The following scientific papers were cited in the current application:
Austin M B, Bowman M E, Ferrer J L, Schroder J, Noel J P. An aldol switch discovered in stilbene synthases mediates cyclization specificity of type III polyketide synthases. Chem Biol. 2004 September; 11(9):1179-94
Ban Z, Qin H, Mitchell A J, Liu B, Zhang F, Weng J K, Dixon R A, Wang G. Noncatalytic chalcone isomerase fold proteins in Humulus lupulus are auxiliary components in prenylated flavonoid biosynthesis. Proc Natl Acad Sci USA. 2018 May 29; 115(22):E5223-E5232.
Burke C and Croteau R. Geranyl diphosphate synthase from Abies grandis: cDNA isolation, functional expression, and characterization. Archives of Biochemistry and Biophysics, Volume 405, Issue 1, 2002, Pages 130-136.
Carvalho A, Hansen E H, Kayser O, Carlsen S, Stehle F. Designing microorganisms for heterologous biosynthesis of cannabinoids. FEMS Yeast Res. 2017 Jun. 1; 17(4).
Chicca A, Schafroth M A, Reynoso-Moreno I, Erni R, Petrucci V, Carreira E M, Gertsch J. Uncovering the psychoactivity of a cannabinoid from liverworts associated with a legal high. Sci Adv. 2018 Oct. 24; 4(10):eaat2166
Christianson T W, Sikorski R S, Dante M, Shero J H, Hieter P. Multifunctional yeast high-copy-number shuttle vectors. Gene. 1992 Jan. 2; 110(1):119-22.
de Groot J C, Weidner C, Krausze J, Kawamoto K, Schroeder F C, Sauer S, Büssow K. Structural characterization of amorfrutins bound to the peroxisome proliferator-activated receptor γ. J Med Chem. 2013 Feb. 28; 56(4):1535-43.
Eckermann C, Schroder G, Eckermann S, Strack D, Schmidt J, Schneider B, Schroder J. Stilbenecarboxylate biosynthesis: a new function in the family of chalcone synthase-related proteins. Phytochemistry. 2003 February; 62(3):271-86.
Eichenberger M, Lehka B J, Folly C et al. Metabolic engineering of Saccharomyces cerevisiae for de novo production of dihydrochalcones with known antioxidant, antidiabetic, and sweet tasting properties. Metab Eng 2017; 39:80-9.
Eichenberger M, Hansson A, Fischer D, Durr L, Naesby M. De novo biosynthesis of anthocyanins in Saccharomyces cerevisiae. FEMS Yeast Res. 2018 Jun. 1; 18(4).
Gagne S J, Stout J M, Liu E, Boubakir Z, Clark S M, Page J E. Identification of olivetolic acid cyclase from Cannabis sativa reveals a unique catalytic route to plant polyketides. Proc Natl Acad Sci USA. 2012 Jul. 31; 109(31):12811-6
Hanuš L O, Meyer S M, Munoz E, Taglialatela-Scafati O, Appendino G. Phytocannabinoids: a unified critical inventory. Nat Prod Rep. 2016 Nov. 23; 33(12):1357-1392.
Kallscheuer N, Menezes R, Foito A, da Silva M H, Braga A, Dekker W, Sevillano D M, Rosado-Ramos R, Jardim C, Oliveira J, Ferreira P, Rocha I, Silva A R, Sousa M, Allwood J W, Bott M, Faria N, Stewart D, Ottens M, Naesby M, Nunes Dos Santos C, Marienhagen J. Identification and Microbial Production of the Raspberry Phenol Salidroside that Is Active against Huntington's Disease. Plant Physiol. 2019 March; 179(3):969-985.
Luttik M A, Vuralhan Z, Suir E, Braus G H, Pronk J T, Daran J M. Alleviation of feedback inhibition in Saccharomyces cerevisiae aromatic amino acid biosynthesis: quantification of metabolic impact. Metab Eng. 2008 May-July; 10(3-4):141-53.
Luo X, Reiter M A, d'Espaux L, Wong J, Denby C M, Lechner A, Zhang Y, Grzybowski A T, Harth S, Lin W, Lee H, Yu C, Shin J, Deng K, Benites V T, Wang G, Baidoo E E K, Chen Y, Dev I, Petzold O, Keasling J D. Complete biosynthesis of cannabinoids and their unnatural analogues in yeast. Nature. 2019 March; 567(7746):123-126.
Mark R, Lyu X, Lee J J L, Parra-Saldivar R, Chen W N. Sustainable production of natural phenolics for functional food applications. Journal of Functional Foods, Volume 57, 2019, Pages 233-254.
Mikkelsen M D, Buron L D, Salomonsen B et al. Microbial production of indolylglucosinolate through engineering of a multi-gene pathway in a versatile yeast expression platform. Metab Eng 2012; 14:104-11.
Mumberg D, Müller R, Funk M. Yeast vectors for the controlled expression of heterologous proteins in different genetic backgrounds. Gene. 1995 Apr. 14; 156(1):119-22.
Rico J, Pardo E, Orejas M. Enhanced production of a plant monoterpene by overexpression of the 3-hydroxy-3-methylglutaryl coenzyme A reductase catalytic domain in Saccharomyces cerevisiae. Appl Environ Microbiol. 2010 October; 76(19):6449-54.
Russo E B. Taming T H C: potential cannabis synergy and phytocannabinoid-terpenoid entourage effects. Br J Pharmacol. 2011 August; 163(7):1344-64.
Sauer S. Amorfrutins: A promising class of natural products that are beneficial to health. Chembiochem. 2014 Jun. 16; 15(9):1231-8.
Schachtsiek J, Warzecha H, Kayser O, Stehle F. Current Perspectives on Biotechnological Cannabinoid Production in Plants. Planta Med. 2018 March; 84(4):214-220.
Shao Z, Zhao H, Zhao H. DNA assembler, an in vivo genetic method for rapid construction of biochemical pathways. Nucleic Acids Res. 2009 February; 37(2):e16.
Shibuya M, Nishioka M, Sankawa U, Ebizuka Y. Incorporation of three deuterium atoms excludes intermediacy of stilbenecarboxylic acid in stilbene synthase reaction. Tetrahedron Letters. 2002 July; 43(29):5071-5074
Sikorski R S, Hieter P. A system of shuttle vectors and yeast host strains designed for efficient manipulation of DNA in Saccharomyces cerevisiae. Genetics 1989; 122:19-27.
Taura F, Iijima M, Yamanaka E, Takahashi H, Kenmoku H, Saeki H, Morimoto S, Asakawa Y, Kurosaki F, Morita H. A Novel Class of Plant Type III Polyketide Synthase Involved in Orsellinic Acid Biosynthesis from Rhododendron dauricum. Front Plant Sci. 2016 September; 7:1452
Vanegas K G, Larsen A B, Eichenberger M, Fischer D, Mortensen U H, Naesby M. Indirect and direct routes to C-glycosylated flavones in Saccharomyces cerevisiae. Microb Cell Fact. 2018 July; 17(1):107. Erratum in: Microb Cell Fact. 2018 Jul. 28; 17(1):119.
Weidner C, de Groot J C, Prasad A, Freiwald A, Quedenau C, Kliem M, Witzke A, Kodelja V, Han C T, Giegold S, Baumann M, Klebl B, Siems K, Müller-Kuhrt L, Schürmann A, Schuler R, Pfeiffer A F, Schroeder F C, Büssow K, Sauer S. Amorfrutins are potent antidiabetic dietary natural products. Proc Natl Acad Sci USA. 2012 May 8; 109(19):7257-62.
Yang X, Matsui T, Kodama T, Mori T, Zhou X, Taura F, Noguchi H, Abe I, Morita H. Structural basis for olivetolic acid formation by a polyketide cyclase from Cannabis sativa. FEBS J. 2016 March; 283(6):1088 106
Zirpel B, Stehle F, Kayser O. Production of Δ9-tetrahydrocannabinolic acid from cannabigerolic acid by whole cells of Pichia (Komagataella) pastoris expressing Δ9-tetrahydrocannabinolic acid synthase from Cannabis sativa L. Biotechnol Lett. 2015 September; 37(9):1869-75.
Hussain T, Plunkett B, Ejaz M, Espley R V and Kayser O. Identification of Putative Precursor Genes for the Biosynthesis of Cannabinoid-Like Compound in Radula marginata. Front. Plant Sci. (2018); 9:537.
Pluskal T, Torrens-Spence M P, Fallon T R et al. The biosynthetic origin of psychoactive kavalactones in kava. Nat. Plants (2019); 5,867-878
Waki T, Takahashi S, and Nakayama T. Managing enzyme promiscuity in plant specialized metabolism: A lesson from flavonoid biosynthesis. BioEssays, (2020); e2000164.

### Sequences

The present application contains a Sequence Listing prepared in PatentIn ver 3.5 submitted electronically in ST25 format which is hereby incorporated by reference in its entirety. The following sequences are included:

| SEQ ID | | TYPE | Code | | Spcecies | Database Acc No. |
|---|---|---|---|---|---|---|
| SEQ ID NO: | Amino acid | Phenylalanine | AtPAL2 | From | Arabidopsis thaliana | NP_190894 |
| 1 | sequence of | ammonia lyase | | | | |
| SEQ ID NO: | Amino acid | Cinnamate 4- | At4CL2 | From | Arabidopsis thaliana | NP_188761 |
| 2 | sequence of | hydroxylase | | | | |
| SEQ ID NO: | Amino acid | Double bond reductase | ScTSC13 | From | Saccharomyces cerevisiae | NP_010269 |
| 3 | sequence of | | | | | |
| SEQ ID NO: | Amino acid | Polyketide synthase | CsTKS | From | Cannabis sativa | BAG14339 |
| 4 | sequence of | | | | | |
| SEQ ID NO: | Amino acid | Polyketide synthase | VvVST1 | From | Vitis vinifera | ABC84860 |
| 5 | sequence of | | | | | |
| SEQ ID NO: | Amino acid | Ployketide synthase | VpSTS1 | From | Vitis pseudoreticulata | ABF06883 |
| 6 | sequence of | | | | | |
| SEQ ID NO: | Amino acid | Polyketide synthase | AhSTS | From | Arachis hypogaea | BAA78617 |
| 7 | sequence of | | | | | |
| SEQ ID NO: | Amino acid | Polyketide synthase | PdSTS2 | From | Pinus densiflora | BAA89667 |
| 8 | sequence of | | | | | |
| SEQ ID NO: | Amino acid | Polyketide synthase | PstSTS2 | From | Pinus strobus | CAA87013 |
| 9 | sequence of | | | | | |
| SEQ ID NO: | Amino acid | Polyketide synthase | PsySTS | From | Pinus sylvestris | CAA43165 |
| 10 | sequence of | | | | | |
| SEQ ID NO: | Amino acid | Tetraketide Cyclase | CsTKC | From | Cannabis sativa | 16WU39 |
| 11 | sequence of | | | | | |
| SEQ ID NO: | Amino acid | HMG-COA reductase | Sc-tHMGR1 | From | Saccharomyces cerevisiae | NP_013636 |
| 12 | sequence of | | | | | |
| SEQ ID NO: | Amino acid | Prenyl Transferase | CsPT1 | From | Cannabis sativa | DAC76711 |
| 13 | sequence of | | | | | |
| SEQ ID NO: | Amino acid | Prenyl Transferase | CsPT3 | From | Cannabis sativa | DAC76713 |
| 14 | sequence of | | | | | |
| SEQ ID NO: | Amino acid | Prenyl Transferase | CsPT4 | From | Cannabis sativa | DAC76710 |
| 15 | sequence of | | | | | |
| SEQ ID NO: | Amino acid | Prenyl Transferase | CsPT5 | From | Cannabis sativa | DAC76714 |
| 16 | sequence of | | | | | |
| SEQ ID NO: | Amino acid | Prenyl Transferase | CsPT7 | From | Cannabis sativa | DAC76716 |
| 17 | sequence of | | | | | |
| SEQ ID NO: | Amino acid | Prenyl Transferase | HIPT1-L | From | Humulus lupulus | A0A0B5A051 |
| 18 | sequence of | | | | | |
| SEQ ID NO: | Amino acid | Prenyl Transferase | HIPT1 | From | Humulus lupulus | E5RP65 |
| 19 | sequence of | | | | | |
| SEQ ID NO: | Amino acid | Prenyl Transferase | HIPT2 | From | Humulus lupulus | A0A0B4ZTQ2 |
| 20 | sequence of | | | | | |
| SEQ ID NO: | Amino acid | Tetrahydrocannabinolic | CsTHCAS | From | Cannabis sativa | Q8GTB6 |
| 21 | sequence of | acid synthase | | | | |
| SEQ ID NO: | Amino acid | Cannabidiolic acid | CsCBDAS | From | Cannabis sativa | A6P6V9 |
| 22 | sequence of | synthase | | | | |
| SEQ ID NO: | Amino acid | CHI-like polypeptide | CsCHIL | From | Cannabis sativa | AFN42529 |
| 23 | sequence of | | | | | |
| SEQ ID NO: | Amino acid | CHI-like polypeptide | HICHIL2 | From | Humulus lupulus | AVR53897 |
| 24 | sequence of | | | | | |
| SEQ ID NO: | Amino acid | Tyrosine ammonia lyase | ZmTAL | From | Zea mays | AAL40137 |
| 25 | sequence of | | | | | |
| SEQ ID NO: | Amino acid | Cinnamate 4- | AmC4H | From | Ammi majus | AAO62904 |
| 26 | sequence of | hydroxylase | | | | |
| SEQ ID NO: | Amino acid | Cytochrome p450 | ScCPR | From | Saccharomyces cerevisiae | BAA02936 |
| 27 | sequence of | reductase | | | | |

### EXAMPLES

### Materials and Methods

### Materials

Chemicals used in the examples herein e.g. for buffers and substrates are commercial products of at least reagent grade.

### Strains and Genes

The Saccharomyces cerevisiae S288C, strain NCYC 3608, is available from the National Collection of Yeast Cultures (NCYC), Norwich, U.K. The LEU2 and HIS3 open reading frames were deleted to create two additional auxotrophies for leucine and histidine, respectively, and the KanMX cassette was excised by Cre-Lox recombination (Eichenberger et al., Met. Eng., 2017, 39: 80-89). The host ARO3 gene was then replaced by feedback insensitive mutants of ARO4 and ARO7 to increase the pool of aromatic amino acids (Luttik et al. 2008; Kallscheuer et al. 2018). The resulting strain, named BBA1, was used as the basic strain in the following examples.

All pathway genes/polynucleotides referred to and disclosed herein, encoding the enzymes and proteins used (SEQ ID NOS: 1-23), were manufactured synthetically by a commercial supplier using codons optimized for expression in yeast, S. cerevisiae, except for ScTSC13 (SEQ ID NO: 3), which was amplified by PCR from yeast genomic DNA. During synthesis, all genes were appended with the DNA sequence AAGCTTAAA at the 5′-end, including a Hind III restriction recognition site and a Kozak sequence, and with the DNA sequence CCGCGG at the 3′-end, including a Sac II recognition site. The gene sequences were maintained and amplified in a multicopy plasmid (pUC18) in E. coli. These plasmids already comprised expression cassettes, including promoters and terminators, previously described by Shao et al. (Nucl. Acids Res. 2009, 37(2):e16), separated by a multiple cloning site, comprising Hind III and Sac II, for easy cloning of the DNA encoding the relevant enzymes.

### Assays and REAGENTS

Bibenzyls (BB), tetraketides (TK), bibenzylic acids (BBA), and all other derivatives were analyzed using liquid-chromatography coupled to mass spectrometry (LC/MS). An HSS T3 column (Waters AG, Baden-Dättwil, Switzerland), 130 Å, 1.7 μm, 2.1 mm×100 mm was employed using the conditions indicated in table 2 below. The following solution were used:

Solution A=0.1% aqueous solution of formic acid,
Solution B=0.1% solution of formic acid in acetonitrile.

TABLE 2
Chromatographic gradient for LCMS analysis

| | Flow | | |
|---|---|---|---|
| Time (min) | (mL/min) | % A | % B |
| Initial | 0.400 | 95.0 | 5.0 |
| 3.00 | 0.400 | 80.0 | 20.0 |
| 4.30 | 0.400 | 80.0 | 20.0 |
| 9.00 | 0.400 | 55.0 | 45.0 |
| 11.00 | 0.400 | 0.0 | 100.0 |
| 13.00 | 0.400 | 0.0 | 100.0 |
| 13.01 | 0.400 | 95.0 | 5.0 |
| 15.00 | 0.400 | 95.0 | 5.0 |

For mass spectrum (MS) analysis, full scan spectrum data were recorded using a Xevo® G2-XS Mass spectrometer (Waters Cooperation, Milford, US) with the parameters indicated in table 3, below.

TABLE 3
Mass spectrometry parameters.

| Source Parameter | Value | |
|---|---|---|
| Ion Source | Electrospray Positive Mode (ESI−) | |
| Capillary Voltage | 2.0 | kV |
| Sampling Cone | 40 | V |
| Source Offset | 80 | V |
| Source Temperature | 150° | C. |
| Desolvation Temperature | 500° | C. |
| Cone gas flow | 100 | L/h |
| De | 1000 | L/h |

| | |
|---|---|
| solvation gas flow | |
| Mass Range | From 50 to 1200 m/z |
| Lock Mass | Leucin Enkephalin (ESI+) |

For each compound, an extracted ion chromatogram within a mass window of 0.01 Da was calculated. Peak areas and compound quantities were calculated according to the retention time and linear calibration curve of the respective standard compounds (obtained from Sigma-Aldrich, Switzerland and/or Extrasynthese, Genay, France) wherever available.

### Culturing Recombinant Host Cells to Produce the Polyketide Compounds of the Invention.

For demonstrating successful production in an engineered yeast via the various heterologous biosynthetic pathways, genes encoding recombinant enzymes were integrated and expressed in the yeast strain. Further, the yeast strain had been engineered to improve the precursor pool of aromatic amino acids, and in some cases the pool of isoprenoids IPP and DMAPP, according to state of the art as described in the scientific literature.

For culturing the engineered yeast strain, cultures of the strain were grown in 96 well, deep well plates (DWP) at 30° C., using 5 cm shaking diameter, and 300 rpm. Pre-cultures were grown for 24 hours from single colonies in 300 μL SC dropout medium (Formedium, Hunstanton, UK), as required for selection of plasmids. The SC dropout medium contained:

### 1.47 g/L Synthetic Complete (Kaiser) Drop Out: Leu, His, Ura (Formedium, Hunstanton, UK),

6.7 g/L Yeast Nitrogen Base without Amino Acids,

### 20 g/L D-(+)-Glucose,

76 mg/L histidine, 380 mg/L leucine, and
76 mg/L uracil depending on the auxotrophies of the strains.
pH of the medium was adjusted to 5.8 with hydrochloric acid.

Main cultures were inoculated in 300 μL of the same medium to a 1:100 dilution of the pre-culture and cultured for 72 hours at 30° C., in 96 well, 1.1 mL deep well plates (DWP) as described by Eichenberger et al. (FEMS Yeast Res. 2018, 1: 18(4)). After 72 hours all cultures had reached essentially the same final optical density (OD) measured at 600 nm. It was contemplated that all compounds of interest were located both intra- and extracellularly and estimates of product titers was based on extraction of total culture volumes.

For extraction of the polyketides and derivatives, 150 μL culture broth was mixed with 150 μL acidified methanol (1% hydrochloric acid) and incubated for 30 min in a 96 well DWP at 30° C., 5 cm shaking diameter, and 300 rpm and subsequently clarified by centrifugation at 4000 g for 5 min. The clarified lysates were analyzed by LC-MS.

### Example 1—Testing a Collection of Polyketide Synthase Enzymes for Production of Bibenzyls

It was tested if a stilbene synthase (STS) type PKS enzyme was able to convert dihydro-cinnamoyl-CoA into a bibenzyl (BB). The genes needed to produce the dihydro-cinnamoyl-CoA precursors, i.e. the Arabidopsis thaliana genes AtPAL2 (SEQ ID NO: 1) and At4CL2 (SEQ ID NO: 2), and the native double bond reductase (DBR) gene ScTSC13 (SEQ ID NO: 3), each under the control of strong glycolytic promoter were first integrated, into the BBA1 host strain. The genes were cloned using homologous recombination technology (HRT) plasmids as described by Eichenberger et al. 2017, thus providing them with recombination tags, promoter and terminator sequences, and the appropriate restriction sites for excision of the expression cassettes (Garcia-Vanegas et al. 2018). The three gene constructs, i.e. the expression cassettes, were integrated into the site XI-3 (Mikkelsen et al. 2012) of strain BBA1 by in vivo homologous recombination as described by Eichenberger et al. 2018, and the production of dihydrocinnamoyl-CoA was confirmed.

A selection of 7 polyketide synthase (PKS) enzymes (See Table 4) were cloned into a yeast expression vector, based on the pRS series of plasmids (Sikorski & Hieter 1989; Christianson et al., Gene 110 (1992) 119-122; Mumberg et al. Gene. 1995 Apr. 14; 156(1):119-22.). Included was the PKS from Cannabis sativa, CsTKS, reported to be a tetraketide synthase (Gagne et al. 2012), whereas the other six were previously reported to be stilbene synthases (STS), from various plants. Stilbene synthases are normally involved in producing bibenzyls with a non-saturated double bond, as known from the compounds pinosylvin and resveratrol, but are known to also accept the reduced dihydro-phenylpropanoic precursors (Eichenberger et al. 2017). The plasmids containing the PKS enzymes, and an empty plasmid as control, were introduced into BBA1, and grown as described above.

TABLE 4
PKS enzymes tested for ability to produce bibenzyls

| | Enzyme | | |
|---|---|---|---|
| SEQ ID NO: | name | Source | Protein acc. no. |
| SEQ ID NO: 4 | CsTKS | Cannabis sativa | BAG14339 |
| SEQ ID NO: 5 | VvVST1 | Vitis vinifera | ABC84860 |
| SEQ ID NO: 6 | VpSTS1 | Vitis pseudoreticulata | ABF06883 |
| SEQ ID NO: 7 | AhSTS | Arachis hypogaea | BAA78617 |
| SEQ ID NO: 8 | PdSTS2 | Pinus densiflora | BAA89667 |
| SEQ ID NO: 9 | PstSTS2 | Pinus strobus | CAA87013 |
| SEQ ID NO: 10 | PsySTS | Pinus sylvestris | CAA43165 |

After 72 hours of growth the total culture medium was extracted and analysed for new products. Both pinosylvin and dihydropinosylvin were detected in various amounts. The occurrence of both the saturated and non-saturated bibenzyl is a reflection of the incomplete reduction, by ScTSC13, of cinnamoyl-CoA to dihydro-cinnamoyl-CoA, leaving both substrates available for the PKS enzymes. The ratio between the two products is a reflection of enzyme substrate specificity, and the total amount of product is a reflection of their efficiency of converting the substrates to bibenzyls.

In all samples an additional compound was detected which had the mass corresponding to the calculated formula weight (276.28) of a free dihydro-cinnamoyl-tetraketide. It was concluded that this compound was the result of dihydro-cinnamoyl-tetraketide-CoA being released from the PKS enzyme, and then hydrolysed to yield the non-activated (no CoA) free tetraketide. Partial hydrolysis could possibly have happened during extraction.

### Example 2—Cyclization of Free Tetraketide by the Tetraketide Cyclase (TKC) from Cannabis sativa

It was tested if olivetolic acid cyclase (CsOAC; SEQ ID NO: 11) was able of accepting tetraketides released from STS-type PKS enzymes, and derived from dihydro-phenylpropanoids, despite these substrates being very bulky. The CsOAC gene was cloned into a second pRS vector (Mumberg et al. 1995). When the CsOAC was co-expressed with STS-type PKS enzymes (see Table 4) in the BBA1 strain, a compound with a mass corresponding to the calculated formula weight (258.27) of the cyclized bibenzylic acid (BBA) was detected, in this case the dihydro-pinosylvin-2-carboxylate. All tested PKS enzymes, including the CsTKS, allowed production of this BBA molecule and, interestingly, the relative amounts produced by the various PKS enzymes corresponded reasonably well with the amount of dihydro-pinosylvin produced in the absence of CsOAC. The highest amount of BBA was achieved when CsOAC was co-expressed, in strain BBA1, with the STS-type PKS enzyme PstSTS2 from Pinus strobus (SEQ ID NO: 9).

### Example 3—Geranylation of Bibenzylic Acid

For testing if the newly formed BBA could be prenylated (FIG. 3) the host strain BBA1 was further modified with the aim of improving the precursor supply of C10 isoprene units in the host. Hence, the native farnesyl pyrophosphate synthetase (ScERG20) was downregulated by replacing the native ERG20 promoter with the weaker native yeast promoter of the ScKEX2 gene, as described in US Patent Application 20180080054. In addition, the geranyl pyrophosphate synthase (GPPS) from Abies grandis (Burke and Croteau, 2002) was overexpressed on a pRS vector. The new strain was named BBA2.

A biosynthetic pathway to prenylated BBAs was then assembled in BBA2, using homologous recombination technology (HRT) plasmids as described in Eichenberger et al. 2017. The plasmids comprised the PstSTS2 (SEQ ID NO: 9) and the CsOAC (SEQ ID NO: 11), as well as a truncated version (Sc-tHMGR; SEQ ID NO: 12) of the native ScHMGR1 gene (Rico et al. 2010). Further, the plasmids comprised one of 8 different prenyltransferases (PT)—(see Table 5). The selection of prenyl transferases was based on sequence homology to the CsPT4 which had previously been shown to transfer geranyl to olivetolic acid, a key step towards cannabinoid production (Luo et al. 2019). After in vivo assembly of 9 plasmids, comprising PstSTS2, ScOAC, Sc-tHMGR, and either a prenyltransferase or an empty cassette, single recombinant colonies were selected and grown for 72 hours with appropriate auxotrophic selection, before being analysed for production of prenylated BBAs.

TABLE 5
Selection of prenyltransferases from Cannabis sativa and Humulus lupulus

| SEQ ID NO: | Enzyme name | Source | Protein acc. no. |
|---|---|---|---|
| SEQ ID NO: 13 | CsPT1 | Cannabis sativa | DAC76711 |
| SEQ ID NO: 14 | CsPT3 | Cannabis sativa | DAC76713 |
| SEQ ID NO: 15 | CsPT4 | Cannabis sativa | DAC76710 |
| SEQ ID NO: 16 | CsPT5 | Cannabis sativa | DAC76714 |
| SEQ ID NO: 17 | CsPT7 | Cannabis sativa | DAC76716 |
| SEQ ID NO: 18 | HlPT1L | Humulus lupulus | A0A0B5A051 |
| SEQ ID NO: 19 | HlPT-1 | Humulus lupulus | E5RP65 |
| SEQ ID NO: 20 | HlPT-2 | Humulus lupulus | A0A0B4ZTQ2 |

The strain comprising the full-length pathway, and including the CsPT4 (SEQ ID NO: 15), produced a new compound with a mass corresponding to the calculated formula weight (394.503) of geranylated BBA, i.e. the bibenzylgerolic acid (BBGA, FIG. 3). BBGA was not found in any of the other strains analysed, i.e. with any other prenyltransferase or without prenyltransferase. It was concluded that CsPT4 was able to geranylate the BBA precursor molecule in yeast. This strain, BBA2 comprising the CsPT4, was named BBA2-PT4. It is contemplated that other prenyltransferases will have a similar activity, e.g. such as prenyltransferases derived from liverworts, in particular those of the genera Radula and Marchantia. Several Radula species are known to produce a variety of prenylated compounds (Hanus et al. 2016) including perrottetinene (PET) (Chicca et al. 2019).

### Example 4—Cyclization of BBGA by THCAS or CBDAS

In order to test whether the BBGA could be further modified by genes of the cannabinoid pathway, expression of CsTHCAS (SEQ ID NO: 21) and CsCBDAS (SEQ ID NO: 22) was tested in yeast. In the strain BBA2-PT4 (see example 3), comprising the PstSTS2 (SEQ ID NO: 9), CsOAC (SEQ ID NO: 11), the truncated HMGR (Sc-tHMGR; SEQ ID NO: 12), and the CsPT4 (SEQ ID NO: 15), a pRS series plasmid (Mumberg et al. 1995) was introduced comprising one of the genes from Cannabis sativa, the CsTHCAS (SEQ ID NO: 21) or the CsCBDAS (SEQ ID NO: 22). In the strain, co-expressing the CsTHCAS a new compound was detected with a mass corresponding to the calculated formula weight (392.487) of perrottetinoic acid (PETA, FIG. 3). Neither a control strain, with an empty pRS plasmid, nor the strain expressing CsCBDAS exhibited any new compounds. In the strain, co-expressing the CsCBDAS a new compound was detected with a mass corresponding to the calculated formula weight (392.487) of perrottetinoic acid (PTDA, FIG. 3). Neither a control strain, with an empty pRS plasmid, nor the strain expressing CsTHCAS exhibited any new compounds.

### Example 5—CHIL Improves the Efficiency of Geranylation

In the strain BBA2-PT4 (see example 3), comprising the PstSTS2 (SEQ ID NO: 9), CsOAC (SEQ ID NO: 11), the truncated HMGR (Sc-tHMGR; SEQ ID NO: 12), and the CsPT4 (SEQ ID NO: 15), a pRS series plasmid (Mumberg et al. 1995) comprising the CsCHIL (SEQ ID NO: 23) from Cannabis sativa or the HICHIL2 (SEQ ID NO: 24) from Humulus lupulus was further introduced. In the strain, co-expressing the HICHIL2 (SEQ ID NO: 24) the production of prenylated bibenzylgerolic acid (BBGA) was higher than in a control strain, with no CHIL protein. It was concluded that some CHIL proteins, including HICHIL2 (SEQ ID NO: 24) are able to assist and improve the production of BBA and BBGA. It is contemplated that other CHIL proteins have similar effects on production of BBA and its derivatives, as well as of related bibenzylic compounds. This includes CHIL proteins derived from plants known to produce prenylated bibenzyls, such as CHIL proteins derived from liverworts, in particular those of the genera Radula and Marchantia. Several Radula species are known to produce perrottetinene (PET, FIG. 3) (Chicca et al. 2019).

1. A genetically modified host cell producing a bibenzylic acid or a derivative thereof expressing:
a) one or more genes encoding a polyketide synthase (PKS);
b) one or more genes encoding a polyketide cyclase (PKC); and
c) one or more genes encoding a double bond reductase (DBR); and one or more genes encoding polypeptides selected from:
d) a tyrosine ammonia lyase polypeptide (TAL);
e) a phenylalanine ammonia lyase polypeptide (PAL);
f) a cinnamate 4-hydroxylase polypeptide (C4H);
g) a cytochrome p450 reductase polypeptide (CPR);
h) a 4-coumarate-CoA ligase polypeptide (4CL); and/or
i) a non-catalytic chalcone isomerase type III or IV polypeptide (CHIL);
wherein the at least one gene is heterologous to the host cell.

2. (canceled)

3. The cell according to claim 1, wherein the double bond reductase is a native enoyl reductase, overexpressed at least two-fold, or a heterologous reductase, capable of reducing the alkene C2-C3 double bond of a phenylpropanoid or phenylpropanoyl-CoA precursor.

4. The cell according to claim 1, wherein
a) the double bond reductase comprises an amino acid sequence which is at least 70%, such as at least 80%, such as at least 90%, such as at least 95%, such as at least 96%, such as at least 97%, such as at least 98%, such as at least 99%, such as 100% identical to the double bond reductase comprised in SEQ ID NO: 3;
b) the polyketide synthase comprises an amino acid sequence which is at least 70%, such as at least 80%, such as at least 90%, such as at least 95%, such as at least 96%, such as at least 97%, such as at least 98%, such as at least 99%, such as 100% identical to the polyketide synthase comprised in SEQ ID NO: 4, 5, 6, 7, 8, 9, or 10;
c) the polyketide cyclase comprises an amino acid sequence which is at least 70%, such as at least 80%, such as at least 90%, such as at least 95%, such as at least 96%, such as at least 97%, such as at least 98%, such as at least 99%, such as 100% identical to the polyketide cyclase comprised in SEQ ID NO: 11;
d) the polyketide cyclase is an olivetolic acid cyclase comprised in SEQ ID NO. 11;
e) the tyrosine ammonia lyase comprises an amino acid sequence which is at least 70%, such as at least 80%, such as at least 90%, such as at least 95%, such as at least 96%, such as at least 97%, such as at least 98%, such as at least 99%, such as 100% identical to the tyrosine ammonia lyase comprised in SEQ ID NO: 25;
f) the phenylalanine ammonia lyase comprises an amino acid sequence which is at least 70%, such as at least 80%, such as at least 90%, such as at least 95%, such as at least 96%, such as at least 97%, such as at least 98%, such as at least 99%, such as 100% identical to the phenylalanine ammonia lyase comprised in SEQ ID NO: 1;
g) the cinnamate 4-hydroxylase comprises an amino acid sequence which is at least 70%, such as at least 80%, such as at least 90%, such as at least 95%, such as at least 96%, such as at least 97%, such as at least 98%, such as at least 99%, such as 100% identical to the cinnamate 4-hydroxylase comprised in SEQ ID NO: 26;
h) the cytochrome p450 reductase comprises an amino acid sequence which is at least 70%, such as at least 80%, such as at least 90%, such as at least 95%, such as at least 96%, such as at least 97%, such as at least 98%, such as at least 99%, such as 100% identical to the cytochrome p450 reductase comprised in SEQ ID NO: 27;
i) the cytochrome p450 reductase comprises an amino acid sequence which is at least 70%, such as at least 80%, such as at least 90%, such as at least 95%, such as at least 96%, such as at least 97%, such as at least 98%, such as at least 99%, such as 100% identical to the cytochrome p450 reductase comprised in SEQ ID NO: 27;
j) the 4-coumarate-CoA ligase comprises an amino acid sequence which is at least 70%, such as at least 80%, such as at least 90%, such as at least 95%, such as at least 96%, such as at least 97%, such as at least 98%, such as at least 99%, such as 100% identical to the 4-coumarate-CoA ligase comprised in SEQ ID NO: 2; and/or
k) the non-catalytic chalcone isomerase like polypeptide comprises an amino acid sequence which is at least 65%, such as at least 70%, such as at least 80%, such as at least 90%, such as at least 95%, such as at least 96%, such as at least 97%, such as at least 98%, such as at least 99%, such as 100% identical to the non-catalytic chalcone isomerase like polypeptide comprised in SEQ ID NO: 23 or 24.

5-6. (canceled)

7. The cell according to claim 1, wherein the polyketide synthase (PKS) produces a linear tetraketide, and/or a free activated linear tetraketide-CoA, optionally from a dihydro-phenylpropanoid, and wherein the PKS has no or limited capability to cyclize the tetraketide, neither by C1-C6 Claisen condensation, nor C2-C7 aldol condensation, optionally by means of an inactivating mutation.

8-15. (canceled)

16. The cell according to claim 1, wherein the bibenzylic acid (BBA) is defined by Formula (I): 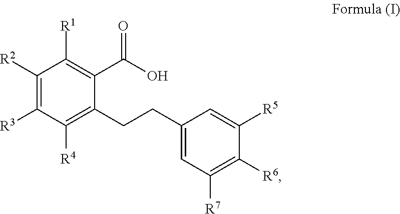
or defined by Formula (II): 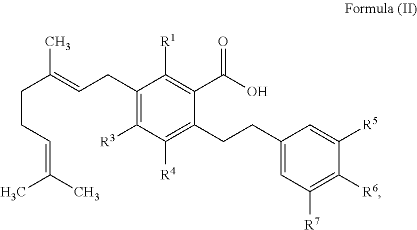
or defined by Formula (III): 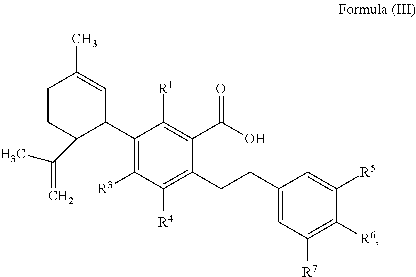
or defined by Formula (IV): 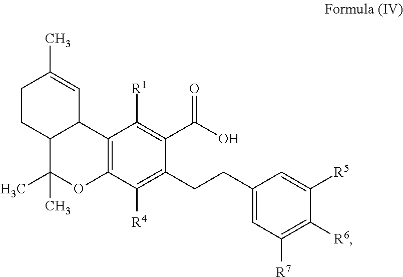
wherein R1, and R4-R7 can be either —H, —OH, —OCH3, —COOH, an acyl group, or a prenyl group.

17. The cell according to claim 16, wherein the cell further expresses a gene encoding a prenyl transferase capable of transferring a prenyl group, such as a dimethylallyl-group, an isopentenyl-group, a geranyl-group, a farnesyl-group, or a geranylgeranyl-group to a bibenzylic acid.

18-19. (canceled)

20. The cell according to claim 17, wherein the prenyl transferase comprises an amino acid sequence which is at least 70%, such as at least 80%, such as at least 90%, such as at least 95%, such as at least 96%, such as at least 97%, such as at least 98%, such as at least 99%, such as 100% identical to the prenyl transferase comprised in SEQ ID NO: 15.

21. The cell according to claim 17, wherein the cell further expresses a gene encoding a synthase converting a compound of Formula II, into a compound, as defined by formula III, having an amino acid sequence which is at least 70%, such as at least 80%, such as at least 90%, such as at least 95%, such as at least 96%, such as at least 97%, such as at least 98%, such as at least 99%, such as 100% identical to the synthase comprised in SEQ ID NO: 22.

22. (canceled)

23. The cell according to claim 17, wherein the cell further expresses a gene encoding a synthase converting a compound of Formula II into a compound, as defined by formula IV, having an amino acid sequence which is at least 70%, such as at least 80%, such as at least 90%, such as at least 95%, such as at least 96%, such as at least 97%, such as at least 98%, such as at least 99%, such as 100% identical to the synthase comprised in SEQ ID NO: 21.

24. (canceled)

25. The cell according to claim 1, wherein the cell is selected from the genera consisting of Saccharomyces, Schizosaccharomyces, Yarrowia, Candida, Ashbya, Cyberlindnera, Pichia, Kluyveromyces, Hansenula, Arxula, and Xanthophyllomyces, optionally from the species Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Kluyveromyces marxianus, Hansenula polymorpha, Candida boidinii, Arxula adeninivorans, Xanthophyllomyces dendrorhous, and Candida albicans.

26. A cell culture, comprising the cell according to claim 1 and a growth medium.

27. A method of producing a BBA or a derivative thereof, comprising:
a) culturing the cell culture of claim 26 at conditions allowing the host cells to produce the BBA or a derivative thereof; and
b) optionally recovering and/or isolating the BBA or a derivative thereof.

28. The method of claim 27, further comprising feeding one or more exogenous precursors for BBA or a derivative thereof to the host cell culture.

29. (canceled)

30. The method of claim 27, wherein the recovering and/or isolation step comprises separating a liquid phase of the cell culture from a solid phase of the cell culture to obtain a supernatant comprising the BBA or a derivative thereof and subjecting the supernatant to one or more steps selected from:
a) contacting the supernatant with one or more adsorbent resins in order to obtain at least a portion of the produced BBA or a derivative thereof, then optionally recovering the BBA or a derivative thereof from the resin in a concentrated solution prior to isolation of the BBA or a derivative thereof by crystallisation or solvent evaporation;
b) contacting the supernatant with one or more ion exchange or reversed-phase chromatography columns in order to obtain at least a portion of the BBA or a derivative thereof, then optionally recovering the BBA or a derivative thereof from the resin in a concentrated solution prior to isolation of the BBA or a derivative thereof by crystallisation or solvent evaporation;
c) extracting the BBA or a derivative thereof from the supernatant, such as by liquid-liquid extraction into an immiscible solvent, then optionally isolating the BBA or a derivative thereof by crystallisation or solvent evaporation; and
thereby recovering and/or isolating the BBA or a derivative thereof.

31. The method of claim 27, wherein one or more steps of producing the BBA or a derivative thereof is performed in vitro.

32. The method of claim 27, further comprising mixing the BBA or a derivative thereof with one or more carriers, agents, adjuvants, additives and/or excipients, optionally pharmaceutical grade carriers, agents, adjuvants, additives and/or excipients.

33. (canceled)

34. A fermentation composition comprising the cell culture of claim 26, wherein at least 50%, such as at least 75%, such as at least 95%, such as at least 99% of the genetically modified host cells are lysed and/or disintegrated, the cell culture further comprising BBA or a derivative thereof, optionally in the form of a dimer.

35. The fermentation composition of claim 34, wherein at least 50%, such as at least 75%, such as at least 95%, such as at least 99% of solid cellular material has separated from the composition.

36-39. (canceled)

40. The composition of claim 34, refined into a dietary supplement, a cosmetic, a food preparation, a feed preparation and/or an analytical or diagnostic reagent.

41. The composition of claim 34 for use as a signal modulator of the cannabinoid receptor 1 (CB1), the cannabinoid receptor 2 (CB2) and/or the PPARgamma receptor.
